(19) United States
(12) Patent Application Publication
(10) Pub. No.: US 2023/0341138 A1
(43) Pub. Date: Oct. 26, 2023

(54) Filter Layer Using Antimicrobial Light

(71) Applicant: Vyv, Inc., Latham, NY (US)

(72) Inventors: Cori J. Winslow, Rensselaer, NY (US); Jonas Ciemny, Colonie, NY (US); Robert Barron, Boulder, CO (US)

(73) Assignee: Vyv, Inc., Latham, NY (US)

(21) Appl. No.: 18/138,487

(22) Filed: Apr. 24, 2023

Related U.S. Application Data
(60) Provisional application No. 63/334,235, filed on Apr. 25, 2022.

(51) Int. Cl. F24F 8/22; F24F 8/108

(52) U.S. Cl. CPC F24F 8/22; F24F 8/108

(57) ABSTRACT

A method and system for disinfecting air purification and heating, ventilation, air conditioning (HVAC) devices may include a fibrous media filter, an antimicrobial filter layer positioned adjacent to one or more surfaces of the fibrous media filter, and one or more light emitters positioned within the antimicrobial filter layer and configured to emit a disinfecting light. The disinfecting light may include an irradiance sufficient to inactivate microorganisms on the fibrous media filter, and the disinfecting light may include a wavelength from about 380 nm to about 420 nm.

### CROSS REFERENCE TO RELATED APPLICATION(S)

This is a utility application claiming priority to and incorporating by reference provisional application entitled Filter Layer Using Antimicrobial Light, Ser. No. 63/334,235 filed Apr. 25, 2022.

### TECHNICAL FIELD

Aspects of the present disclosure generally relate to processes, systems, and apparatus for a filter layer using antimicrobial light.

### BACKGROUND

Air purification devices and systems and/or devices utilizing filters for air filtration, are subject to microorganism build up within the devices, specifically on the filter surfaces. Microorganisms trapped in filters, such as fibrous media filters, may continue to grow and replicate on and within the filter surfaces. Microbes may have the ability to remain alive for an extended period on the filter, especially if trapped along with enriched particulate matter and/or moisture that create environments facilitating growth of microorganisms. Many fibrous media filters, which are intended to filter out items such as dust, pollen, lint, hair, animal fur, etc., do not comprise pores small enough to trap microorganisms. The microbes may eventually pass through the filter and be redistributed into the environment. There are known negative impacts to human lungs from breathing in air contaminated with microorganisms and, additionally, odor can be spread through an environment as a result of microorganism build up. When air purification devices or systems using filters remain off for an extended period, the lack of air movement may improve the environment for microorganism growth and replication. As a result, when the device is powered back on and air pushes through the fibrous media filter again, it provides an additional opportunity for microbes to be redistributed into the environment.

Microorganism inactivation is a crucial practice required in many areas of both personal and environmental hygiene for the benefit of human health. Many methods are employed for a variety of situations where human or animal health factors may be improved by inactivation of bacteria, viruses, and other microorganisms. Sickness and infection are the primary concerns of microorganism contamination through the many modes of intake of organisms into the human body from the environment, including from air purification or HVAC (heating, ventilation, air conditioning) devices. The human body may become sickened or infected by many different modes. Some modes may be due to internal imbalances of natural human microorganisms, but many problematic cases are caused by the transmission of microorganisms by either human to human contact or proximity, or by intake of microorganisms from the immediate environment, including breathing in contaminated air.

It would be desirable to eliminate or destroy harmful microorganisms contaminating the environments of air purification and HVAC devices to benefit human and animal health. In particular, it would be advantageous to passively sanitize the system filters of air purification and HVAC devices, as well as building and housing interiors, product interiors, surfaces, etc.

### SUMMARY

The following presents a simplified summary in order to provide a basic understanding of some aspects of the disclosure. The summary is not an extensive overview of the disclosure. It is neither intended to identify key or critical elements of the disclosure nor to delineate the scope of the disclosure. The following summary merely presents some concepts of the disclosure in a simplified form as a prelude to the description below.

In accordance with aspects disclosed herein, a system for sanitizing air purification and HVAC devices may include a fibrous media filter, an antimicrobial filter layer positioned adjacent to one or more surfaces of the fibrous media filter, and one or more light emitters positioned within the antimicrobial filter layer and configured to emit a disinfecting light comprising an irradiance sufficient to inactivate microorganisms on the fibrous media filter. In some examples, the disinfecting light may comprise a wavelength from about 380 nm to about 420 nm.

In accordance with other aspects disclosed herein, a method of sanitizing air purification and HVAC devices may include the steps of providing a fibrous media filter in the air purification or HVAC device, positioning an antimicrobial filter layer adjacent to one or more surfaces of the fibrous media filter, embedding one or more light emitters within the antimicrobial filter layer, wherein the one or more light emitters are configured to emit a disinfecting light comprising an irradiance sufficient to inactivate microorganisms, illuminating the one or more surfaces of the fibrous media filter with the disinfecting light of the one or more light emitters, and inactivating microorganisms on the fibrous media filter. In some examples, the disinfecting light may comprise a wavelength from about 380 nm to about 420 nm.

The foregoing and other features of this disclosure will be apparent from the following description of examples of the disclosure.

### BRIEF DESCRIPTION OF THE DRAWINGS

FIG. 1A illustrates the frame portion of the antimicrobial filter layer disclosed herein.

FIG. 1B is a front facing view of FIG. 1A.

FIG. 1C illustrates the frame portion of the antimicrobial filter layer with the linear light modules placed within the frame as disclosed herein.

FIG. 1D is a zoomed in version of FIG. 1C.

FIG. 2A illustrates the back portion of the antimicrobial filter layer that houses the internal components as disclosed herein.

FIG. 2B is a front facing view of FIG. 2A.

FIG. 2C illustrates the frame populated with light modules and the associated wiring as disclosed herein.

FIG. 2D is a zoomed in version of FIG. 2C.

FIG. 3A illustrates an example of a wire connector used to provide power to the antimicrobial filter layer as disclosed herein.

FIG. 3B is a perspective view of FIG. 3A.

FIG. 4A illustrates an example light module as disclosed herein.

FIG. 4B is a perspective view of the light module of FIG. 4A.

FIG. 5A illustrates another example light module as disclosed herein.

FIG. 5B is a perspective view of the light module of FIG. 5A.

FIG. 6A illustrates an example lens mounted over the light module and in the frame as disclosed herein.

FIG. 6B is a perspective view of FIG. 6A without the frame.

FIG. 6C illustrates a back/rear view of an example lens mounted over the light module without the frame as disclosed herein.

FIG. 6D illustrates an example of an isolated lens as disclosed herein.

FIG. 7 illustrates another example of an isolated lens as disclosed herein.

FIG. 8 illustrates another example lens design mounted on an example frame as disclosed herein.

FIG. 9A illustrates an example cover placed over an example frame with individual lenses mounted over each light emitter as disclosed herein.

FIG. 9B illustrates another example cover placed over an example frame with individual lenses mounted over each light emitter or linear light module as disclosed herein.

FIG. 10A illustrates an isolated cover example as disclosed herein.

FIG. 10B is a front view of the frame of FIG. 10A.

FIG. 11 illustrates an assembled antimicrobial filter layer without lenses allowing visualization of the placement of the light modules as disclosed herein.

FIG. 12 illustrates a full assembled antimicrobial filter layer including lenses, cover, light modules, frame, and required wiring components as disclosed herein.

FIG. 13A illustrates the antimicrobial filter layer containing a fibrous media filter placed in position against standoffs as disclosed herein.

FIG. 13B is a side view of FIG. 13A.

FIG. 13C illustrates an alternate view of FIG. 13A facing the air intake side.

FIG. 14A illustrates the addition of a pre-filter coupled to the antimicrobial filter layer that is offset from the fibrous media filter as disclosed herein.

FIG. 14B is a side view of FIG. 14A.

FIG. 14C illustrates a pre-filter coupled to the air intake side of the antimicrobial filter layer as disclosed herein.

FIG. 14D illustrates the antimicrobial filter layer with the addition of the fibrous media filter placed in position against standoffs as disclosed herein.

FIG. 15 illustrates a pre-filter as disclosed herein.

FIG. 16A illustrates an adsorbent media filter coupled to the fibrous media filter as disclosed herein.

FIG. 16B is a side view of FIG. 16A.

FIG. 17A illustrates the use of an antimicrobial filter layer on each side of a fibrous media filter as disclosed herein.

FIG. 17B is a side view of FIG. 17A.

FIG. 18A illustrates the addition of a pre-filter on the air intake side of an assembly using an antimicrobial filter layer on each side of a fibrous media filter as disclosed herein.

FIG. 18B illustrates the addition of an adsorbent media milter on the assembly of FIG. 18A.

FIG. 18C is a side view of FIG. 18B.

FIG. 19A illustrates an example antimicrobial filter layer with two sides providing illumination and combined into a single device with a location at the bottom to position a fibrous media filter as disclosed herein.

FIG. 19B illustrates FIG. 19A with the fibrous media filter in place.

FIG. 19C illustrates addition of a pre-filter and an adsorbent media filter to the example antimicrobial filter layer of FIG. 19B.

FIG. 19D is a top view of FIG. 19C.

FIG. 20A illustrates an example frame for use with a cylindrical fibrous media filter as disclosed herein.

FIG. 20B is a top view of FIG. 20A.

FIG. 21 illustrates linear light modules positioned within frame channels as disclosed herein.

FIG. 22A illustrates the addition of lenses over the channels comprised the linear light modules.

FIG. 22B is a top view of FIG. 22A.

FIG. 23A illustrates the addition of a cover over a frame as disclosed herein.

FIG. 23B illustrates a top view of FIG. 23A.

FIG. 24A illustrates a cylindrical fibrous media filter positioned within the antimicrobial filter layer as disclosed herein.

FIG. 24B is a bottom-perspective view of FIG. 24A.

FIG. 24C is a top view of FIG. 24A.

FIG. 25A illustrates a frame for an antimicrobial filter layer and further including an additional inner core of channels for linear light modules as disclosed herein.

FIG. 25B is a top view of FIG. 25A.

FIG. 26 illustrates the frame of FIG. 25A with linear light modules installed within the channels of the exterior of the frame and the inner core as disclosed herein.

FIG. 27A illustrates a full antimicrobial filter layer including an additional inner core for providing illumination into the fibrous media filter as disclosed herein.

FIG. 27B is a top view of FIG. 27A.

FIG. 28A illustrates a cylindrical fibrous media filter positioned within the antimicrobial filter layer as disclosed herein.

FIG. 28B is a bottom perspective view of FIG. 28B.

FIG. 28C is a top view of FIGS. 28A and 28B.

FIG. 29A illustrates the addition of an adsorbent media filter wrapped around the outside of an antimicrobial filter layer disclosed herein.

FIG. 29B is a top view of FIG. 29A.

FIG. 30A illustrates an antimicrobial filter layer with a cover positioned over the top blocking air from passing through a frame gap as disclosed herein.

FIG. 30B is a bottom perspective view of FIG. 30A.

FIG. 31 illustrates a pre-filter coupled to an antimicrobial filter layer as disclosed herein.

FIG. 32 illustrates an adsorbent media filter wrapped around the outside of an antimicrobial filter layer comprising a built-in pre-filter as disclosed herein.

FIG. 33 is a cross-sectional view of FIG. 32.

FIG. 34 schematically depicts the intensity of a disinfecting light from a light emitter based upon the angle the disinfecting light is emitted from the light emitter.

FIG. 35A illustrates another example cylindrical frame including a larger inner core for the placement of an adsorbent media filter within the inner core as disclosed herein.

FIG. 35B is a top view of FIG. 35A.

FIG. 36 illustrates the example antimicrobial filter layer of FIG. 35A with the fibrous media filter and adsorbent media filter in position.

FIG. 37A illustrates the addition of a cover onto the assembly of FIG. 36.

FIG. 37B is a cross-sectional view of FIG. 37A.

### DETAILED DESCRIPTION

In the following description of the various embodiments, reference is made to the accompanying drawings, which form a part hereof, and in which is shown by way of illustration, various embodiments of the disclosure that may be practiced. It is to be understood that other embodiments may be utilized.

Wavelengths of visible light in the violet range, 380-420 nanometer (nm) (e.g., 405 nm), may have a lethal effect on microorganisms. As used herein, the term “microorganisms” encompasses at least viruses (including enveloped and non-enveloped viruses), bacteria (including gram positive and gram negative bacteria), bacterial endospores, yeasts, molds, and filamentous fungi. For example, Escherichia coli (E. coli), Salmonella, Methicillin-resistant Staphylococcus aureus (MRSA), and Clostridium difficile may be susceptible to 380-420 nm light. Such wavelengths may initiate a photoreaction within non-iron porphyrin molecules found in some microorganisms. The non-iron porphyrin molecules may be photoactivated and may react with other cellular components to produce Reactive Oxygen Species (ROS). ROS may cause irreparable cell damage and eventually destroy, kill, or otherwise inactivate cells of some microorganisms. Non-iron porphyrins are specific to microorganisms only therefore, because humans, plants, and/or animals do not contain these same non-iron porphyrin molecules, this technique may be completely safe for human, plant, and animal exposure. Light in the 380-420 nm wavelength may be effective against every type of bacteria, although it may take different amounts of time or dosages depending upon the species. Light with a 380-420 nm wavelength (e.g., 405 nm), may be effective against all gram-negative and gram-positive bacteria to some extent over a period of time. It can also be effective against many varieties of fungi.

In some examples, visible light in the violet range, 380-420 nanometer (nm) (e.g., 405 nm), may decrease viral load on a surface. Viruses may rely on surface bacteria, yeast, mold, or fungi as hosts. By decreasing surface bacteria, yeast, mold, or fungi count, for example, by using 380-420 nm light, the viral load may also be decreased. In some examples, viruses may be susceptible to reactive oxygen species. Viral load may decrease when the viruses are surrounded by a medium that can produce reactive oxygen species to inactivate viruses. In some examples, the medium may comprise fluids or droplets that comprise bacteria or other particles that produce oxygen reactive species. In some examples, the medium may comprise respiratory droplets, saliva, feces, organic rich media, and/or blood plasma.

In some examples, inactivation, in relation to microorganism death, may include control and/or reduction in microorganism colonies or individual cells when exposed to disinfecting light for a certain duration. Light may be utilized for inactivation using a peak wavelength of light, or in some examples, multiple peak wavelengths, in a range of approximately 380 nm to 420 nm. For example, approximately 405 nm light may be used as the peak wavelength. It should be understood that any wavelength within 380 nm to 420 nm may be utilized, and that the peak wavelength may include a specific wavelength plus or minus approximately 5 nm. According to one example, peak wavelength may include, for example, at least, greater than, less than, equal to, or any number in between about 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, 393 nm, 394 nm, 395 nm, 396 nm, 397 nm, 398 nm, 399 nm, 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, and 425 nm. Such light may damage viral capsids, surface proteins, nucleic acids, and also lead to the degradation of the nucleic acids. Destruction of nucleic acids and genomes may prevent replication function in host cells leading to loss of infectivity. Unsaturated lipids and alterations of envelope proteins may cause conformational changes in the viral structure that alters viral interactions with host cell receptors. Protein mediated binding, injection or replication functions may be impaired. Significant changes in molecular mass and charge of proteins may occur, which may hinder viral entry and cytopathic effects.

The electromagnetic spectrum may be harnessed within devices, systems, and apparatuses to utilize its functions for benefit of humans/animals. Most portions of the electromagnetic spectrum are not visible with the exception of the visible light spectrum within the range of approximately 380 nm to 750 nm. The ultraviolet spectrum comprises the energy within the range of approximately 100 nm to 400 nm and is generally not visible. Light comprising wavelengths that provide microorganisms inactivation or disinfection may be referred to as “disinfecting light.” Disinfecting light may be emitted by one or more light emitters.

There may be a minimum irradiance required to hit the surface to cause microbial inactivation. A target irradiance may be required on at least a portion of the surface. A minimum irradiance of light (e.g., in the 380-420 nm wavelength) on a surface may cause microbial inactivation. For example, a minimum irradiance of 0.02 milliwatts per square centimeter (mW/cm²) may cause microbial inactivation on a surface over time. In some examples, an irradiance of 0.05 mW/cm² may inactivate microorganisms on a surface, but higher values such as 0.1 mW/cm², 0.5 mW/cm², 1 mW/cm², or 2 mW/cm² may be used for quicker microorganism inactivation. In some examples, even higher irradiances may be used over shorter periods of time, e.g., 3 to 10 mW/cm². In other examples, a target irradiance may be, for example, at least, greater than, less than, equal to, or any number in between about 0.01 mW/cm², 0.02 mW/cm², 0.03 mW/cm², 0.04 mW/cm², 0.05 mW/cm², 0.06 mW/cm², 0.07 mW/cm², 0.08 mW/cm², 0.09 mW/cm², 0.1 mW/cm², 0.1 mW/cm², 0.2 mW/cm², 0.3 mW/cm², 0.4 mW/cm², 0.5 mW/cm², 0.6 mW/cm², 0.7 mW/cm², 0.8 mW/cm², 0.9 mW/cm², 1.0 mW/cm², 1.1 mW/cm², 1.2 mW/cm², 1.3 mW/cm², 1.4 mW/cm², 1.5 mW/cm², 1.6 mW/cm², 1.7 mW/cm², 1.8 mW/cm², 1.9 mW/cm², 2.0 mW/cm², 2.1 mW/cm², 2.2 mW/cm², 2.3 mW/cm², 2.4 mW/cm², 2.5 mW/cm², 2.6 mW/cm², 2.7 mW/cm², 2.8 mW/cm², 2.9 mW/cm², 3.0 mW/cm², 3.1 mW/cm², 3.2 mW/cm², 3.3 mW/cm², 3.4 mW/cm², 3.5 mW/cm², 3.6 mW/cm², 3.7 mW/cm², 3.8 mW/cm², 3.9 mW/cm², 4.0 mW/cm², 4.1 mW/cm², 4.2 mW/cm², 4.3 mW/cm², 4.4 mW/cm², 4.5 mW/cm², 4.6 mW/cm², 4.7 mW/cm², 4.8 mW/cm², 4.9 mW/cm², 5.0 mW/cm², 5.1 mW/cm², 5.2 mW/cm², 5.3 mW/cm², 5.4 mW/cm², 5.5 mW/cm², 5.6 mW/cm², 5.7 mW/cm², 5.8 mW/cm², 5.9 mW/cm², 6.0 mW/cm², 6.1 mW/cm², 6.2 mW/cm², 6.3 mW/cm², 6.4 mW/cm², 6.5 mW/cm², 6.6 mW/cm², 6.7 mW/cm², 6.8 mW/cm², 6.9 mW/cm², 7.0 mW/cm², 7.1 mW/cm², 7.2 mW/cm², 7.3 mW/cm², 7.4 mW/cm², 7.5 mW/cm², 7.6 mW/cm², 7.7 mW/cm², 7.8 mW/cm², 7.9 mW/cm², 8.0 mW/cm², 8.1 mW/cm², 8.2 mW/cm², 8.3 mW/cm², 8.4 mW/cm², 8.5 mW/cm², 8.6 mW/cm², 8.7 mW/cm², 8.8 mW/cm², 8.9 mW/cm², 9.0 mW/cm², 9.1 mW/cm², 9.2 mW/cm², 9.3 mW/cm², 9.4 mW/cm², 9.5 mW/cm², 9.6 mW/cm², 9.7 mW/cm², 9.8 mW/cm², 9.9 mW/cm², and 10.0 mW/cm². Example light emitters disclosed herein may be configured to produce light with such irradiances at any given surface.

In some examples, an average irradiance is targeted across a surface or at least a portion of a surface. The average may comprise an average of multiple measurement points taken across at least a portion of the surface. Irradiance measurements may range from 0 mW/cm² to 100 mW/cm² in some examples. In some examples, the target average irradiance may be 0.05 mW/cm². In some examples, the target average irradiance may be 1 mW/cm². In some examples, the target average irradiance may be any value within the range of 0.02 to 2 mW/cm². In some examples, the target average irradiance may be any value within the range of 0.02 to 5 mW/cm². In still another example, the average irradiance may be, for example, at least, greater than, less than, equal to, or any number in between about 0.01 mW/cm², 0.02 mW/cm², 0.03 mW/cm², 0.04 mW/cm², 0.05 mW/cm², 0.06 mW/cm², 0.07 mW/cm², 0.08 mW/cm², 0.09 mW/cm², 0.1 mW/cm², 0.1 mW/cm², 0.2 mW/cm², 0.3 mW/cm², 0.4 mW/cm², 0.5 mW/cm², 0.6 mW/cm², 0.7 mW/cm², 0.8 mW/cm², 0.9 mW/cm², 1.0 mW/cm², 1.1 mW/cm², 1.2 mW/cm², 1.3 mW/cm², 1.4 mW/cm², 1.5 mW/cm², 1.6 mW/cm², 1.7 mW/cm², 1.8 mW/cm², 1.9 mW/cm², 2.0 mW/cm², 2.1 mW/cm², 2.2 mW/cm², 2.3 mW/cm², 2.4 mW/cm², 2.5 mW/cm², 2.6 mW/cm², 2.7 mW/cm², 2.8 mW/cm², 2.9 mW/cm², 3.0 mW/cm², 3.1 mW/cm², 3.2 mW/cm², 3.3 mW/cm², 3.4 mW/cm², 3.5 mW/cm², 3.6 mW/cm², 3.7 mW/cm², 3.8 mW/cm², 3.9 mW/cm², 4.0 mW/cm², 4.1 mW/cm², 4.2 mW/cm², 4.3 mW/cm², 4.4 mW/cm², 4.5 mW/cm², 4.6 mW/cm², 4.7 mW/cm², 4.8 mW/cm², 4.9 mW/cm², 5.0 mW/cm², 5.1 mW/cm², 5.2 mW/cm², 5.3 mW/cm², 5.4 mW/cm², 5.5 mW/cm², 5.6 mW/cm², 5.7 mW/cm², 5.8 mW/cm², 5.9 mW/cm², 6.0 mW/cm², 6.1 mW/cm², 6.2 mW/cm², 6.3 mW/cm², 6.4 mW/cm², 6.5 mW/cm², 6.6 mW/cm², 6.7 mW/cm², 6.8 mW/cm², 6.9 mW/cm², 7.0 mW/cm², 7.1 mW/cm², 7.2 mW/cm², 7.3 mW/cm², 7.4 mW/cm², 7.5 mW/cm², 7.6 mW/cm², 7.7 mW/cm², 7.8 mW/cm², 7.9 mW/cm², 8.0 mW/cm², 8.1 mW/cm², 8.2 mW/cm², 8.3 mW/cm², 8.4 mW/cm², 8.5 mW/cm², 8.6 mW/cm², 8.7 mW/cm², 8.8 mW/cm², 8.9 mW/cm², 9.0 mW/cm², 9.1 mW/cm², 9.2 mW/cm², 9.3 mW/cm², 9.4 mW/cm², 9.5 mW/cm², 9.6 mW/cm², 9.7 mW/cm², 9.8 mW/cm², 9.9 mW/cm², and 10.0 mW/cm².

In some examples, light for microbial inactivation may include radiometric energy sufficient to inactive at least one microorganism population, or in some examples, a plurality of microorganism populations. One or more light emitters(s) may emit some minimum amount of radiometric energy (e.g., 20 mW) measured from 380-420 nm light. In one example, one or more light emitter(s) may emit some minimum amount of radiometric energy measured from, for example, at least, greater than, less than, equal to, or any number in between about 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, 393 nm, 394 nm, 395 nm, 396 nm, 397 nm, 398 nm, 399 nm, 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, and 425 nm.

In another example, one or more light emitter(s) may emit some minimum amount of radiometric energy measured from, for example, at least, greater than, less than, equal to, or any number in between about 10 mW, 15 mW, 20 mW, 25 mW, 30 mW, 35 mW, 40 mW, 45 mW, 50 mW, 55 mW, 60 mW, 65 mW, 70 mW, 75 mW, 80 mW, 85 mW, 90 mW, 95 mW, 100 mW, 105 mW, 110 mW, 115 mW, 120 mW, 125 mW, 130 mW, 135 mW, 140 mW, 145 mW, 150 mW, 155 mW, 160 mW, 165 mW, 170 mW, 175 mW, 180 mW, 185 mW, 190 mW, 195 mW, 200 mW, 205 mW, 210 mW, 215 mW, 220 mW, 225 mW, 230 mW, 235 mW, 240 mW, 245 mW, 250 mW, 255 mW, 260 mW, 265 mW, 270 mW, 275 mW, 280 mW, 285 mW, 290 mW, 295 mW, 300 mW, 305 mW, 310 mW, 315 mW, 320 mW, 325 mW, 330 mW, 335 mW, 340 mW, 345 mW, 350 mW, 355 mW, 360 mW, 365 mW, 370 mW, 375 mW, 380 mW, 385 mW, 390 mW, 395 mW, 400 mW, 405 mW, 410 mW, 415 mW, 420 mW, 425 mW, 430 mW, 435 mW, 440 mW, 445 mW, 450 mW, 455 mW, 460 mW, 465 mW, 470 mW, 475 mW, 480 mW, 485 mW, 490 mW, 495 mW, 500 mW, 505 mW, 510 mW, 515 mW, 520 mW, 525 mW, 530 mW, 535 mW, 540 mW, 545 mW, 550 mW, 555 mW, 560 mW, 565 mW, 570 mW, 575 mW, 580 mW, 585 mW, 590 mW, 595 mW, 600 mW, 605 mW, 610 mW, 615 mW, 620 mW, 625 mW, 630 mW, 635 mW, 640 mW, 645 mW, 650 mW, 655 mW, 660 mW, 665 mW, 670 mW, 675 mW, 680 mW, 685 mW, 690 mW, 695 mW, 700 mW, 705 mW, 710 mW, 715 mW, 720 mW, 725 mW, 730 mW, 735 mW, 740 mW, 745 mW, 750 mW, 755 mW, 760 mW, 765 mW, 770 mW, 775 mW, 780 mW, 785 mW, 790 mW, 795 mW, 800 mW, 805 mW, 810 mW, 815 mW, 820 mW, 825 mW, 830 mW, 835 mW, 840 mW, 845 mW, 850 mW, 855 mW, 860 mW, 865 mW, 870 mW, 875 mW, 880 mW, 885 mW, 890 mW, 895 mW, 900 mW, 905 mW, 910 mW, 915 mW, 920 mW, 925 mW, 930 mW, 935 mW, 940 mW, 945 mW, 950 mW, 955 mW, 960 mW, 965 mW, 970 mW, 975 mW, 980 mW, 985 mW, 990 mW, 995 mW, 1000 mW, 1005 mW, 1010 mW, 1015 mW, 1020 mW, 1025 mW, 1030 mW, 1035 mW, 1040 mW, 1045 mW, 1050 mW, 1055 mW, 1060 mW, 1065 mW, 1070 mW, 1075 mW, 1080 mW, 1085 mW, 1090 mW, 1095 mW, 1100 mW, 1105 mW, 1110 mW, 1115 mW, 1120 mW, 1125 mW, 1130 mW, 1135 mW, 1140 mW, 1145 mW, 1150 mW, 1155 mW, 1160 mW, 1165 mW, 1170 mW, 1175 mW, 1180 mW, 1185 mW, 1190 mW, 1195 mW, 1200 mW, 1205 mW, 1210 mW, 1215 mW, 1220 mW, 1225 mW, 1230 mW, 1235 mW, 1240 mW, 1245 mW, 1250 mW, 1255 mW, 1260 mW, 1265 mW, 1270 mW, 1275 mW, 1280 mW, 1285 mW, 1290 mW, 1295 mW, 1300 mW, 1305 mW, 1310 mW, 1315 mW, 1320 mW, 1325 mW, 1330 mW, 1335 mW, 1340 mW, 1345 mW, 1350 mW, 1355 mW, 1360 mW, 1365 mW, 1370 mW, 1375 mW, 1380 mW, 1385 mW, 1390 mW, 1395 mW, 1400 mW, 1405 mW, 1410 mW, 1415 mW, 1420 mW, 1425 mW, 1430 mW, 1435 mW, 1440 mW, 1445 mW, 1450 mW, 1455 mW, 1460 mW, 1465 mW, 1470 mW, 1475 mW, 1480 mW, 1485 mW, 1490 mW, 1495 mW, 1500 mW, 1505 mW, 1510 mW, 1515 mW, 1520 mW, 1525 mW, 1530 mW, 1535 mW, 1540 mW, 1545 mW, 1550 mW, 1555 mW, 1560 mW, 1565 mW, 1570 mW, 1575 mW, 1580 mW, 1585 mW, 1590 mW, 1595 mW, 1600 mW, 1605 mW, 1610 mW, 1615 mW, 1620 mW, 1625 mW, 1630 mW, 1635 mW, 1640 mW, 1645 mW, 1650 mW, 1655 mW, 1660 mW, 1665 mW, 1670 mW, 1675 mW, 1680 mW, 1685 mW, 1690 mW, 1695 mW, 1700 mW, 1705 mW, 1710 mW, 1715 mW, 1720 mW, 1725 mW, 1730 mW, 1735 mW, 1740 mW, 1745 mW, 1750 mW, 1755 mW, 1760 mW, 1765 mW, 1770 mW, 1775 mW, 1780 mW, 1785 mW, 1790 mW, 1795 mW, 1800 mW, 1805 mW, 1810 mW, 1815 mW, 1820 mW, 1825 mW, 1830 mW, 1835 mW, 1840 mW, 1845 mW, 1850 mW, 1855 mW, 1860 mW, 1865 mW, 1870 mW, 1875 mW, 1880 mW, 1885 mW, 1890 mW, 1895 mW, 1900 mW, 1905 mW, 1910 mW, 1915 mW, 1920 mW, 1925 mW, 1930 mW, 1935 mW, 1940 mW, 1945 mW, 1950 mW, 1955 mW, 1960 mW, 1965 mW, 1970 mW, 1975 mW, 1980 mW, 1985 mW, 1990 mW, 1995 mW, and 2000 mW.

Dosage (measured in Joules/cm²) may be another metric for determining an appropriate irradiance for microbial inactivation over a period of time. Table 1 below shows example correlations between irradiance in mW/cm² and Joules/cm² based on different exposure times. These values are examples and many others may be possible.

TABLE 1

| Irradiance (mW/cm²) | Exposure Time (hours) | Dosage (Joules/cm²) |
|---|---|---|
| 0.02 | 1 | 0.072 |
| 0.02 | 24 | 1.728 |
| 0.02 | 250 | 18 |
| 0.02 | 500 | 36 |
| 0.02 | 1000 | 72 |
| 0.05 | 1 | 0.18 |
| 0.05 | 24 | 4.32 |
| 0.05 | 250 | 45 |
| 0.05 | 500 | 90 |
| 0.05 | 1000 | 180 |
| 0.1 | 1 | 0.36 |
| 0.1 | 24 | 8.64 |
| 0.1 | 250 | 90 |
| 0.1 | 500 | 180 |
| 0.1 | 1000 | 360 |
| 0.5 | 1 | 1.8 |
| 0.5 | 24 | 43.2 |
| 0.5 | 250 | 450 |
| 0.5 | 500 | 900 |
| 0.5 | 1000 | 1800 |
| 1 | 1 | 3.6 |
| 1 | 24 | 86.4 |
| 1 | 250 | 900 |
| 1 | 500 | 1800 |
| 1 | 1000 | 3600 |
| 2 | 1 | 7.2 |
| 2 | 24 | 172.8 |
| 2 | 250 | 1800 |
| 2 | 500 | 3600 |
| 2 | 1000 | 7200 |
| 5 | 1 | 18 |
| 5 | 24 | 432 |
| 5 | 250 | 4500 |
| 5 | 500 | 9000 |
| 5 | 1000 | 18000 |

Microbial inactivation may comprise a target reduction in microorganism population(s) (e.g., 1-Log₁₀ reduction, 2-Log₁₀ reduction, 99% reduction, or the like). Table 2 shows example dosages recommended for the inactivation (measured as 1-Log10 reduction in population) of different microorganism species using narrow spectrum 405 nm light. Example dosages and other calculations shown herein may be determined based on laboratory settings. Real world applications may require dosages that may differ from example laboratory data. Other dosages of 380-420 nm (e.g., 405 nm) light may be used with other bacteria not listed below.

TABLE 2

| | Recommended Dose (J/cm²) for |
|---|---|
| Microorganism | 1-Log Reduction in Microorganism |
| Staphylococcus aureus | 20 |
| MRSA | 20 |
| Pseudomonas aeruginosa | 45 |
| Escherichia coli | 80 |
| Enterococcus faecalis | 90 |

Equation 1 may be used in order to determine irradiance, dosage, or time using one or more data points from Table 1 and Table 2:

$$\frac{Irradiance ( \frac{m W}{c {m}^{2}} )}{1000} * Time ( s ) = Dosage ( \frac{J}{{cm}^{2}} ) \qquad Equation 1$$

Irradiance may be determined based on dosage and time. For example, if a dosage of 30 Joules/cm² is recommended and the object desired to be disinfected is exposed to light overnight for 8 hours, the irradiance may be approximately 1 mW/cm². If a dosage of 50 Joules/cm² is recommended and the object desired to be disinfected is exposed to light for 48 hours, a smaller irradiance of only approximately 0.3 mW/cm² may be sufficient.

Time may be determined based on irradiance and dosage. For example, light emitter(s) may be configured to provide an irradiance of disinfecting energy (e.g., 0.05 mW/cm²) and a target bacteria may require a dosage of 20 Joules/cm² to kill the target bacteria. Disinfecting light at 0.05 mW/cm² may have a minimum exposure time of approximately 4.6 days to achieve the dosage of 20 Joules/cm². Dosage values may be determined by a target reduction in microorganisms. Once the microorganism count is reduced to a desired amount, disinfecting light may be continuously applied to keep the microorganism counts down.

Radiant power (e.g., radiometric power, optical output power, spectral power etc.), measured in Watts, is the total power emitted from a light source. Irradiance is the power per unit area on a surface at a distance away from the light source. In some examples, the target irradiance on a target surface from the light source may be 10 mW/cm². A 10 mW/cm² target irradiance may be provided, for example, by light emitter(s) with a total radiant power of 10 mW located 1 cm from the target surface. In another example, light emitter(s) may be located 5 cm from the target surface. With a target irradiance of 10 mW/cm², the light source may be configured to produce a radiant power approximately 250 mW. These calculations may be approximately based on the inverse square law, as shown in Equation 1, where the excitation light source may be assumed to be a point source, E is the irradiance, I is the radiant power, and r is the distance from the excitation light source to a target surface.

$$E ≅ \frac{I}{{r}^{2}} \qquad Equation 2$$

In some examples, different wavelengths of light may have different effects on different microorganisms. The tables below illustrate example data related to application of various wavelengths of light on various microorganisms. For example, tables 3-7 summarize the recommended dose response for the inactivation of microorganisms at different log levels when exposed to wavelengths of 405 nm, 222 nm and 254 nm light. Inactivation may comprise a target reduction in microorganism population(s) (e.g., 1-Log₁₀ reduction, 2-Log₁₀ reduction, 99% reduction, or the like).

Table 3 shows example dosages measured in J/cm² which may be used for the inactivation (at different log levels) of different microorganisms using 222 nm light.

TABLE 3

| | Recommended Dose (J/cm²) for | | | |
|---|---|---|---|---|
| | Reduction in Microorganisms at 222 nm | | | |
| Microorganism | 1-log | 2-log | 3-log | 4-log |
| Staphylococcus | 9.3 × 10⁻³ | 1.15 × 10⁻² | 1.38 × 10⁻² | 1.78 × 10⁻² |
| aureus | | | | |
| Pseudomonas | 3.1 × 10⁻³ | 4.8 ×10⁻³ | 5.9 × 10⁻³ | 7.5 × 10⁻³ |
| aeruginosa | | | | |
| Aspergillus | 9 × 10⁻² | 0.220 | 0.325 | 0.430 |
| niger | | | | |

Table 4 shows example dosages measured in J/cm² which may be used for the inactivation (at different log levels) of different microorganisms using 254 nm light.

TABLE 4

| | Recommended Dose (J/cm²) for | | | |
|---|---|---|---|---|
| | Reduction in Microorganisms at 254 nm | | | |
| Microorganism | 1-log | 2-log | 3-log | 4-log |
| Staphylococcus | 4.4 × 10⁻³ | 6.0 × 10⁻³ | 7.3 × 10⁻³ | 9.5 × 10⁻³ |
| aureus | | | | |
| Streptococcus | 6.6 × 10⁻³ | 8.8 × 10⁻³ | 9.9 × 10⁻³ | 1.12 × 10⁻² |
| faecalis | | | | |
| Pseudomonas | 8 × 10⁻⁴ | 1.6 × 10⁻³ | 2.3 × 10⁻³ | 3.1 × 10⁻³ |
| aeruginosa | | | | |
| Escherichia coli | 3 × 10⁻³ | 4.8 × 10⁻³ | 6.7 × 10⁻³ | 8.4 × 10⁻³ |
| Aspergillus | 0.115 | 0.245 | 0.370 | 0.560 |
| niger | | | | |

Table 5 shows example dosages measured in J/cm² which may be used for the inactivation (at different log levels) of different microorganisms using 222 nm light.

TABLE 5

| | Recommended Dose (J/cm²) for | | | |
|---|---|---|---|---|
| | Reduction in Microorganisms at 222 nm | | | |
| Microorganism | Type | Reduction | Light dosage | Medium |
| Influenza A | Enveloped | 1 log | 1.3 × 10⁻³ | Airborne |
| | | 2 log | 2.6 × 10⁻³ | |
| | | 3 log | 3.8 × 10⁻³ | |
| HCoV 229-E | Enveloped | 1 log | 5.6 × 10⁻⁴ | Airborne |
| | | 2 log | 1.1 × 10⁻³ | |
| | | 3 log | 1.7 × 10⁻³ | |
| HCoV OCV3 | Enveloped | 1 log | 3.9 × 10⁻⁴ | Airborne |
| | | 2 log | 7.8 × 10⁻⁴ | |
| | | 3 log | 1.2 × 10⁻³ | |

Table 6 shows example dosages measured in J/cm² which may be used for the inactivation (at different log levels) of different microorganisms using 254 nm light.

TABLE 6

| | Type | Reduction | | Light dosage | Medium |
|---|---|---|---|---|---|
| Influenza A | Enveloped | 1 | log | 1.04 × 10⁻³ | Airborne |
| | | 1.4 | log | 1.48 × 10⁻³ | |
| Influenza A | Enveloped | 4.08 log to 5.75 log | | 1.8 | Solid |
| SARS CoV | Enveloped | 3.4 log to 3.6 log | | 0.15 | Liquid |
| | | 4 | log | | |
| SARS CoV | Enveloped | 4 | log | 0.12 | Solid |
| SARS CoV2 | Enveloped | 5.7 | log | 1.6 × 10⁻² | Liquid |
| MS₂ | Non-enveloped | 1 | log | 3.4-4.2 × 10⁻³ | Airborne |
| bacteriophage | | 2 | log | 8-9.1 × 10⁻⁴ | |
| MS₂ | Non-enveloped | 1 | log | 1.86-2.57 × 10⁻² | Liquid |
| bacteriophage | | 4 | log | 0.12 | |
| MS₂ | Non-enveloped | 1 | log | 3.2 × 10⁻³ | Solid |
| bacteriophage | | 3 log to 4 log | | 4.32-7.2 | |
| FCV | Non-enveloped | 1 | log | 5-6 × 10⁻³ | Liquid |
| | | 4 | log | 0.04 | |
| FCV | Non-Enveloped | 2.12 log-4.46 log | | 0.2 | Solid |
| Adenovirus type | Non-enveloped | 1 | log | 5.5 × 10⁻² | Liquid |
| 40 | | 2 | log | 0.105 | |
| | | 3 | log | 0.155 | |
| Rotavirus | Non-enveloped | 1 | log | 2.0 × 10⁻² | Liquid |
| | | 2 | log | 8.0 × 10⁻² | |
| | | 3 | log | 0.140 | |
| | | 4 | log | 0.2 | |
| Polio virus 1 | Non-enveloped | 1 | log | 7 × 10⁻³ | Liquid |
| | | 2 | log | 1.7 × 10⁻² | |
| | | 3 | log | 2.8 × 10⁻² | |
| | | 4 | log | 3.7 × 10⁻² | |
| Hepatitis A | Non-enveloped | 1 | log | 5.5 × 10⁻³ | Liquid |
| | | 2 | log | 9.8 × 10⁻³ | |
| | | 3 | log | 1.5 × 10⁻² | |
| | | 4 | log | 2.1 × 10⁻² | |
| Murine | Non-enveloped | 1 | log | 1 × 10⁻² | Liquid |
| norovirus | | | | | |

Table 7 shows example dosages measured in J/cm² which may be used for the inactivation (at different log levels) of different microorganisms using 405 nm light.

TABLE 7

| | Recommended Dose (J/cm²) for | | | | |
|---|---|---|---|---|---|
| | Reduction in Microorganisms at 405 nm | | | | |
| Microorganism | Type | Reduction | | Light dosage | Medium |
| SARS CoV2 | Enveloped | 1 | log | 3.9 × 10⁻⁴ | Airborne |
| phi6 | Enveloped | 1 | log | 430 | Liquid |
| | | 3 | log | 1300 | |
| Bacteriophage | Non-Enveloped | 3 | log | 300 | Liquid |
| sigma C31 | | 5 | log | 500 | |
| | | 7 | log | 1400 | |
| FCV | Non-enveloped | 3.9 | log | 2800 | Liquid |

In some examples, one or more of the light emitter(s) disclosed herein may inactivate microorganisms/pathogens with light having a peak wavelength of light, or in some examples, multiple peak wavelengths, in a range of approximately 380 nm to approximately 420 nm. For example, approximately 405 nm light may be used as the peak wavelength. It should be understood that any wavelength within 380 nm to 420 nm may be utilized, and that the peak wavelength may include a specific wavelength plus or minus approximately 5 nm. In some examples, one or more light emitter(s) may emit some minimum amount of radiometric energy measured from, for example, at least, greater than, less than, equal to, or any number in between about 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, 393 nm, 394 nm, 395 nm, 396 nm, 397 nm, 398 nm, 399 nm, 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, and 425 nm.

In some examples, one or more of the light emitter(s) disclosed herein may inactivate microorganisms/pathogens with light having a peak wavelength of light, or in some examples, multiple peak wavelengths, in a range of approximately 200 nm to approximately 380 nm, for example, approximately 254 nm light may be used as the peak wavelength. It should be understood that any wavelength within 200 nm to 380 nm may be utilized, and that the peak wavelength may include a specific wavelength plus or minus approximately 5 nm. Light sources may additionally be within the following ranges: 100-280 nm, 200-230 nm, and/or 380-420 nm including, for example, UVA, UVC, visible, 222 nm, 254 nm, 260-270 nm, 280 nm, and/or 405 nm peak wavelength. In another example, one or more of the light emitter(s) disclosed herein may inactivate microorganisms/pathogens with light having a peak wavelength of light, or in some examples, multiple peak wavelengths, in a range of, at least, greater than, less than, equal to, or any number in between about 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, 220 nm, 221 nm, 222 nm, 223 nm, 224 nm, 225 nm, 226 nm, 227 nm, 228 nm, 229 nm, 230 nm, 231 nm, 232 nm, 233 nm, 234 nm, 235 nm, 236 nm, 237 nm, 238 nm, 239 nm, 240 nm, 241 nm, 242 nm, 243 nm, 244 nm, 245 nm, 246 nm, 247 nm, 248 nm, 249 nm, 250 nm, 251 nm, 252 nm, 253 nm, 254 nm, 255 nm, 256 nm, 257 nm, 258 nm, 259 nm, 260 nm, 261 nm, 262 nm, 263 nm, 264 nm, 265 nm, 266 nm, 267 nm, 268 nm, 269 nm, 270 nm, 271 nm, 272 nm, 273 nm, 274 nm, 275 nm, 276 nm, 277 nm, 278 nm, 279 nm, 280 nm, 281 nm, 282 nm, 283 nm, 284 nm, 285 nm, 286 nm, 287 nm, 288 nm, 289 nm, 290 nm, 291 nm, 292 nm, 293 nm, 294 nm, 295 nm, 296 nm, 297 nm, 298 nm, 299 nm, 300 nm, 301 nm, 302 nm, 303 nm, 304 nm, 305 nm, 306 nm, 307 nm, 308 nm, 309 nm, 310 nm, 311 nm, 312 nm, 313 nm, 314 nm, 315 nm, 316 nm, 317 nm, 318 nm, 319 nm, 320 nm, 321 nm, 322 nm, 323 nm, 324 nm, 325 nm, 326 nm, 327 nm, 328 nm, 329 nm, 330 nm, 331 nm, 332 nm, 333 nm, 334 nm, 335 nm, 336 nm, 337 nm, 338 nm, 339 nm, 340 nm, 341 nm, 342 nm, 343 nm, 344 nm, 345 nm, 346 nm, 347 nm, 348 nm, 349 nm, 350 nm, 351 nm, 352 nm, 353 nm, 354 nm, 355 nm, 356 nm, 357 nm, 358 nm, 359 nm, 360 nm, 361 nm, 362 nm, 363 nm, 364 nm, 365 nm, 366 nm, 367 nm, 368 nm, 369 nm, 370 nm, 371 nm, 372 nm, 373 nm, 374 nm, 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, 393 nm, 394 nm, 395 nm, 396 nm, 397 nm, 398 nm, 399 nm, 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, and 425 nm.

In some examples, the device disclosed herein may use continuous disinfection. For example, an object or a surface intended to be disinfected may be continuously irradiated by one or more of the light emitter(s) disclosed herein. In some examples, an object or surface may be illuminated for a first percentage of time (e.g., 80% of the time) and not illuminated for a second percentage of time (e.g., 20% of the time), such as when the object or surface is being interacted with by a human, e.g., when changing a filter, etc. In some examples, an integrated control system may determine that a minimum dosage over a certain period of time has been met for disinfecting purposes and disinfecting light may be turned off to save energy until the period of time expires and resets. In some examples, disinfecting light may be turned off 30% of the time over a specific time period, such as 24 hours, and may still be considered continuous (e.g., 16.8 hours out of 24). Other similar ratios may be possible.

In some examples, the light emitter(s) disclosed herein may use intermittent disinfection. Some examples use intermittent disinfecting techniques where the disinfecting light may be only irradiating an object or surface intended to be disinfected, e.g., a filter, for certain period of time. In some examples, disinfecting light may shine on the object or surface intended to be disinfected for 8 hours overnight. In some examples, disinfecting light may shine on the object or surface intended to be disinfection for a period of time between 30 seconds and 8 hours. In some examples, the period of time the object or surface is exposed to the disinfecting light may match up with a specific time required to meet a certain dosage target for the inactivation of a specific microorganism.

In some examples, one or more of the light sources disclosed herein may pulse disinfecting light. By pulsing the disinfecting light emitter(s) or otherwise reducing its duty cycle below 100%, the dose and exposure may be decreased, and the lifetime of the light emitter(s) may be increased. Pulsed light at high irradiances may be more effective than continuous light at lower irradiances. In some examples, pulsed light may have higher exposure limits compared to a continuous light source. In some examples, pulsed light may be considered to be intermittent because the light will be on and off periodically. In some examples, however, pulsed light may be used continuously and thus may also be considered continuous disinfection due to the length of time that light is pulsed (e.g., light may be pulsed for 24 hours straight).

In some examples, the light emitter(s) may emit light according to a proportion of spectral energy. The proportion of spectral energy may be an amount of spectral energy within a specified wavelength range, i.e., 380-420 nm, divided by a total amount of spectral energy of the light. In some examples, the proportion of spectral energy may be a percentage.

The light emitted from the light emitter(s) may comprise a proportion of a spectral energy of the light, measured in a 380 nanometers (nm) to 420 nm wavelength range, greater than 50%. The light may comprise a full width half max (FWHM) emission spectrum of less than 20 nm and centered at a wavelength of approximately 405 nm to concentrate the spectral energy of the light and minimize energy associated with wavelengths that bleed into an ultraviolet wavelength range. The light may provide an irradiance at the surface sufficient to initiate inactivation of microorganisms on the surface.

Different colors of light may be emitted with a percentage (e.g., 20%) of their spectral energy within the wavelength range of 380-420 nm or within a UV wavelength range. In some examples, various colors of light may be emitted with a percentage of 30% to 100% spectral power within the wavelength range of 380-420 nm. For example, a white light containing light across the visible light spectrum from 380-750 nm, may be used for disinfection purposes, with at least 20% of its energy within the wavelength range of 380-420 nm.

In some examples, light emitted from light emitter(s) may be white, may have a color rendering index (CRI) value of at least 70, may have a correlated color temperature (CCT) between approximately 2,500K and 5,000K and/or may have a proportion of spectral energy measured in the 380 nm to 420 nm wavelength range between 10% and 44%. Other colors (e.g., blue, green, red, etc.) may also be used with a minimum percentage of spectral energy (e.g., 20%) within the range of 380-420 nm, which provides the disinfecting energy. In some examples, the white light may include a proportion of spectral energy measured in the 200 nm to 230 nm wavelength range between 0.01% and 2%.

Light emitter(s) may take any light emitter form capable of emitting light or energy e.g., light emitting diode (LED), LEDs with light-converting layer(s), laser, electroluminescent wires, electroluminescent sheets, flexible LEDs, organic light emitting diode (OLED), or a semiconductor die.

In some examples, the light emitters may be LEDs (light emitting diodes) emitting light with a peak wavelength, for example, at least, greater than, less than, equal to, or any number in between about 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, 393 nm, 394 nm, 395 nm, 396 nm, 397 nm, 398 nm, 399 nm, 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, and 425 nm.

In some examples, light disinfection may be provided for devices to control the growth of harmful microorganisms and prevent illness in humans as well as other negative side effects of microorganisms such as odor or visually unappealing mold and/or fungi. Light disinfection may be provided to fibrous media filters within air purification devices or any device or system using a fibrous media filter, such as a HEPA filter.

Due to the location of fibrous media filters within air purification devices, it may be difficult to effectively illuminate the fibrous media filter surfaces with disinfecting light from light emitter(s). Some devices have tight internal designs with limited gaps for disinfecting light to illuminate fibrous media filter surfaces. Additionally, other filters used around the fibrous media filters such as adsorbent media filters and pre-filters may block disinfecting light from reaching the surface of the fibrous media filter. For this reason, an antimicrobial filter layer may need to be added to the typical filter structure of air purification devices to allow for effective disinfecting illumination directly onto the surfaces of a fibrous media filter.

There are many different types of air purification and HVAC devices that use fibrous media filters. Many consumer, commercial, and industrial air purification processed use fibrous media filters, such as HEPA filters for air purification. Filters use mechanical mechanisms for removing particles from the air. Air purification devices may take the form factor of tower form factors for residential, whole home systems built into the HVAC systems of home and commercial/industrial buildings or built into ceiling structures of operating suites or cleanroom environments for advanced air purification. Some air purification devices also comprise the use of electrical and/or chemical based processes for air purification. Multiple processes may be used within one air purification device, including the disclosed antimicrobial filter layer device.

Pre-filters may be used within air purification devices. Pre-filters often have larger pores or openings that block large particles from entering the device, such as hair.

Adsorbent media filters are often used within air purification devices. These filters use adsorption to neutralize odors. Adsorption occurs when the filter traps odor molecules when they attach to the adsorbent media filter surface. These filters may be carbon based, such as using activated charcoal. Over time, the adsorbent media filter will saturate and require cleaning or to be replaced. In some examples, it is important that adsorbent media filters are easily accessible and removable within air purification devices and systems. Adsorbent media filters may be flexible or rigid and come in multiple different form factors. Adsorbent media filters may comprise an activated carbon or charcoal structure to trap VOCs (volatile organic compounds) and/or odors. Due to the structure of adsorbent media filters, they can also trap larger airborne particles, such as dust, pollen, pet dander, etc.

In some air purification devices, ionizers are used to target particles. Ionizers release ions, which attach to opposite charged airborne particles and cause them to fall to the ground, the bottom surface of a device, or cling to walls, ceilings, or device surfaces. This effectively pulls the particles out of the air.

Photo-catalytic oxidation may be used in some air purification devices. This process targets gasses and VOCs in the air by transforming gaseous pollutants into water and carbon dioxide using TiO₂ in the oxidation process and UV light for activating the TiO₂. In some examples, the activating UV light may also be a disinfecting light used for disinfecting purposes in addition to activating photocatalysts such as TiO₂.

Photoelectrochemical oxidation may be used by air purification devices. This process uses a filter coated with a nano-catalyst activated by UV wavelengths to create hydroxyl radicals which break apart particles trapped in the filter into water and carbon dioxide.

Electrostatic precipitation may be used in some air purification devices. This process ionizes the air by a corona discharge process and collects particles on electrically charged plates.

In some air purification devices, plasma processes may be used to target gas and VOCs by transforming gaseous pollutants by breaking their chemical bond using electrical arcs.

In some air purification devices, ozone generators may be used to produce ozone which breaks down gases and VOCs. Ozone may be produced using UV light wavelengths or corona discharge.

The antimicrobial filter layer may be a device located adjacent to one or more surfaces of a fibrous media filter. The antimicrobial filter layer comprises one or more light emitters emitting wavelengths of disinfecting light. The disinfecting light provides disinfecting illumination on one or more surfaces of the fibrous media filter. An irradiance of disinfecting light may be achieved on and/or within the fibrous media filter layer sufficient to inactivate microorganisms.

The antimicrobial filter layer may be a device located adjacent to any surface within the air purification device or system. The antimicrobial filter layer may be a device located adjacent to an adsorbent media filter or any other type of filter used within a ventilation, HVAC, or air purification device.

In some examples, the antimicrobial filter layer may be a device located adjacent to a grease filter used in range hood ventilation devices.

In some examples, the disinfecting light is directed to one surface of the fibrous media filter, i.e., the surface that becomes more contaminated with microorganisms. This may be on the air intake or air exit/output side of the fibrous media filter. In some examples, the disinfecting light is directed to two or more surfaces of the fibrous media filter. In some examples, this may be both sides of a flat rectangular fibrous media filter. In some examples, this may be the outside and inside of a cylindrical and/or tube shaped filter.

In some examples, the antimicrobial filter layer may be used or combined with other types of filter layers such as adsorbent media filters and/or pre-filters. In some examples, the one or more additional filter layers may be removably coupled to the antimicrobial filter layer. In some examples, the one or more additional filter layers be built into the antimicrobial filter layer. The orientation and order of the filters layers may be different in different embodiments. In some examples, the air may flow in the following order: pre-filter, antimicrobial filter layer, fibrous media filter, adsorbent media filter. In some examples, the air may flow in the following order: pre-filter, adsorbent media filter, antimicrobial filter layer, fibrous media filter. In some examples, the air may flow in the following order: pre-filter, antimicrobial filter layer, fibrous media filter, antimicrobial filter layer, adsorbent media filter. Other arrangements, orders, and orientations are possible. Other filter layers or air purification mechanisms may additionally be included in the air flow process.

The shape, size, and overall geometry of the antimicrobial filter layer may be dependent on the shape, size, and overall geometry of the fibrous media filter it is illuminating. In some examples, fibrous media filters are flat rectangular shapes. In some examples, fibrous media filters and rectangular shapes which a specific depth. In some examples, fibrous media filters are cylindrical in shape with a hollow center, i.e., tube shaped. Other antimicrobial filter layer shapes and sizes are possible.

In some examples, the antimicrobial filter comprises a frame for housing the light emitters. In some examples, the frame may be made of a metal material. In some examples, the frame may be made from a polymer material. In other examples, the frame may be made of a combination of metal and polymer materials. The frame may have large gaps, in a grid pattern for example, to allow for air to pass through. On the side of the frame facing the fibrous media filter, there may be channels for light emitters, light emitters populated onto substrates (“light modules”), and/or electrical wiring. The frame may comprise channels or housing areas for light modules populated with one or more light emitters.

In some examples, the frame may comprise linear light modules comprising one or more light emitters. In some examples, the frame may comprise individual light modules comprising one light emitter.

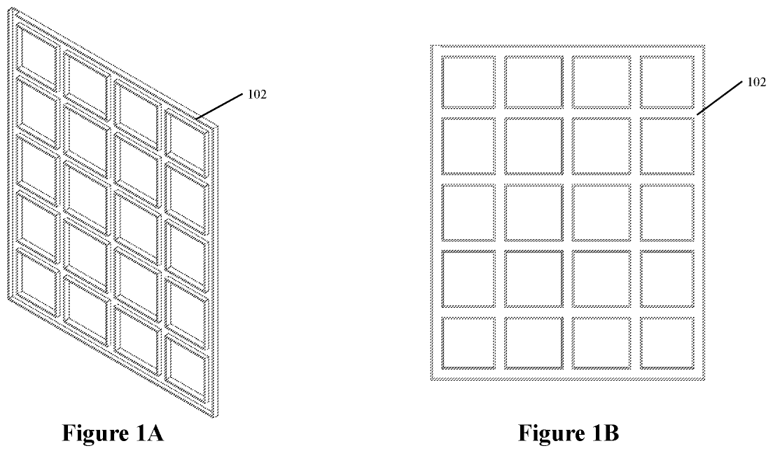

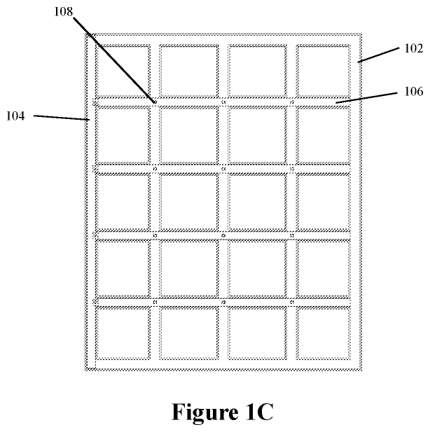

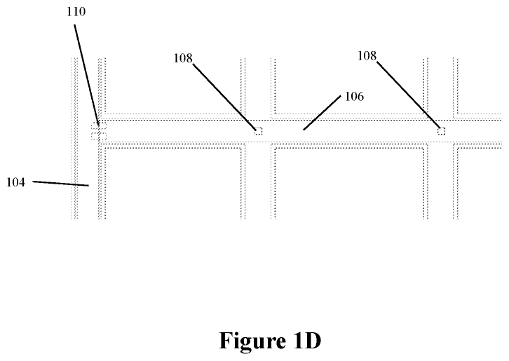

FIGS. 1A and 1B show an example rectangular frame 102 for use with a flat fibrous media filter. FIGS. 1A and 1B comprises channels in a grid pattern to allow for gaps between channels for the air to flow through. FIGS. 1A and 1B show an example frame design that may be used for housing linear light modules.

FIGS. 1C and 1D show example linear light modules 106 installed within the channels of the frame 102. In some examples, the linear light modules 106 are printed circuit boards populated with one or more light emitters. The example of FIGS. 1C and 1D show the use of four linear light modules 106 each populated with three light emitters 108. In other examples, linear light module 106 may include at least 1, 2, 3, 4, 5, 6, 7, 8, 9, 10, 11, or 12 light emitters 108. FIG. 1C additionally shows an example connecting module 104, not populated with light emitters. This connecting module 104 runs perpendicular to the light modules 106 and may receive input power external to the device and be connected to the light modules 106 to provide them power. In other examples, the connecting module 104 may provide solar power or battery power to the light modules 106. In some examples, the connecting module 104 is connected to the light module(s) 106 through wires or by soldering directly to the light module 106. As shown in FIG. 1D, the connecting module 104 may connect to an external power source, and connection point 110 may connect the light module 106. Other light module sizes, shapes, and quantities of light emitters are possible.

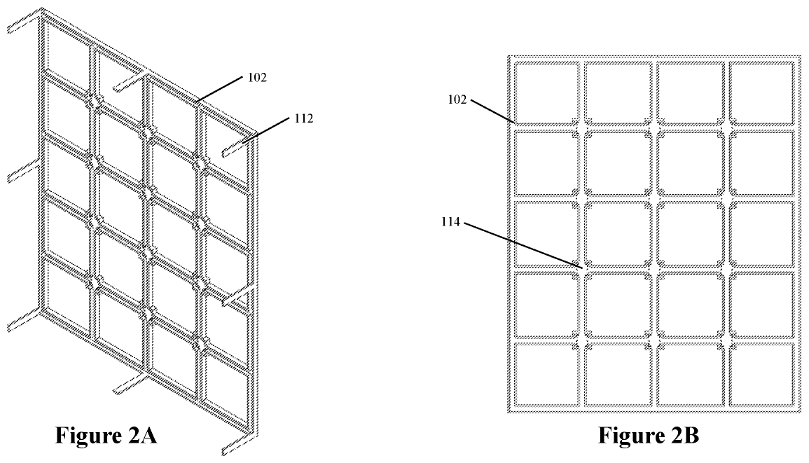

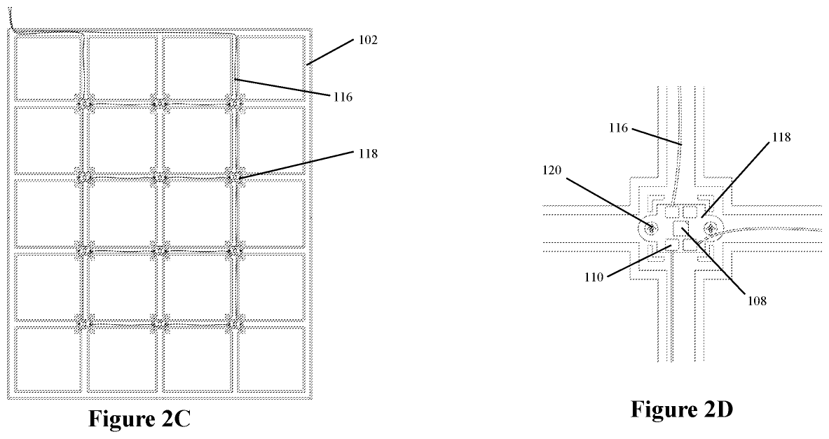

In some examples, the frame may comprise standoffs along the edges that allow for a gap between the antimicrobial filter layer and the fibrous media filter. This gap is required for effective disinfecting illumination onto the fibrous media filter surface. FIG. 2A shows example standoffs 112 built into the frame 102. In some examples this gap may be equal to or greater than 0.25 in. In some examples this gap may be equal to or less than 2 in. In some examples, this gap may be equal to or less than 12 in. In other examples, the gap may be, for example, at least, greater than, less than, equal to, or any number in between about 0.25 in, 0.5 in, 0.75 in, 1 in, 1.25 in, 1.5 in, 1.75 in, 2 in, 2.25 in, 2.5 in, 2.75 in, 3 in, 3.25 in, 3.5 in, 3.75 in, 4 in, 4.25 in, 4.5 in, 4.75 in, 5 in, 5.25 in, 5.5 in, 5.75 in, 6 in, 6.25 in, 6.5 in, 6.75 in, 7 in, 7.25 in, 7.5 in, 7.75 in, 8 in, 8.25 in, 8.5 in, 8.75 in, 9 in, 9.25 in, 9.5 in, 9.75 in, 10 in, 10.25 in, 10.5 in, 10.75 in, 11 in, 11.25 in, 11.5 in, 11.75 in, and 12 in.

In some examples, the light emitters emit at a beam angle of 130 degrees. In other examples, the light emitters emit at a beam angle of, for example, at least, greater than, less than, equal to, or any number in between about 90 degrees, 95 degrees, 100 degrees, 105 degrees, 110 degrees, 115 degrees, 120 degrees, 125 degrees, 130 degrees, 135 degrees, 140 degrees, 145 degrees, 150 degrees, 155 degrees, 160 degrees, 165 degrees, 170 degrees, 175 degrees, 180 degrees. If the gap is approximately 2 in, in some examples, then approximately 12 light emitters spaced approximately evenly in a grad pattern will provide illumination to the entire surface of an approximately 17 in×14 in fibrous media filter. This example is shown for reference only, other filter sizes, shapes, beam angles, light emitter quantities, and gap distances are possible.

In some examples, the frame comprises a housing location for a light module populated with one or more light emitters. FIG. 2B shows an example frame 102 with housing locations 114 for light modules to be placed.

FIG. 2C shows example wiring 116 used to provide power to the light modules 118. The wire 116 may be directly soldered to the light modules 118 or attached to the light modules 118 through connectors populated onto the light module.

FIG. 2D shows a zoomed in image of a singular light module 118 installed in the frame 102. In some examples, the frame 102 may include mounting guides for placing light emitters disposed on substrates. There are multiple methods the light emitter disposed on a substrate may be mounted to the frame 102. In some examples, the substrate may be mounted using hardware such as screws and/or using adhesive. In some examples, the substrate may be mounted with spring clips. In examples where mounting hardware is used for the substrate, mounting holes may be included in the frame. As shown in FIG. 2D, singular light module 118 may be secured to the frame 102 by mounting screw 120. Singular light module 118 may include at least two sets of solder pads, at least one light emitter 108, and multiple connection points 110. Frame 102 may also include standoffs for mounting of the light module 106 or singular light module 118. The shape of light module 118 is an example only, other shapes and/or sizes are possible.

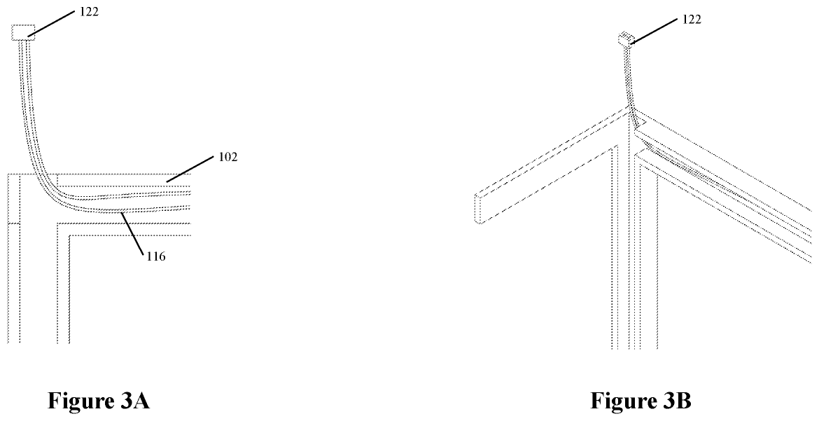

In some examples, a wiring harness exits the frame and comprises a connector for power. FIGS. 3A and 3B show an example wire 116 with connector 122 exiting the frame 102 to apply power to the antimicrobial filter layer. In some examples, the air purification device antimicrobial filter layer will comprise an embedded mating connector 122 to provide power directly from the air purification device. In some examples, the connection 122 from antimicrobial filter layer to power may be a locking connector with male and female pins. In some examples, the connection from antimicrobial filter layer to power may utilize spring loaded contacts to allow for the removal of the antimicrobial filter layer without manual disconnection of electrical power. In some examples, an LED driver is included within or remote from the antimicrobial filter layer to provide the required power to the antimicrobial filter layer.

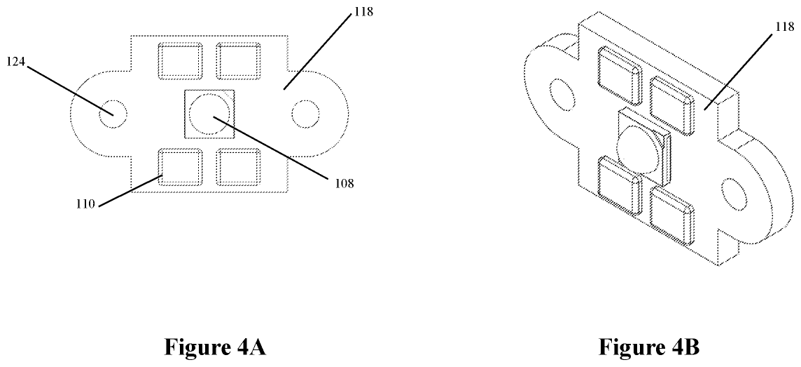

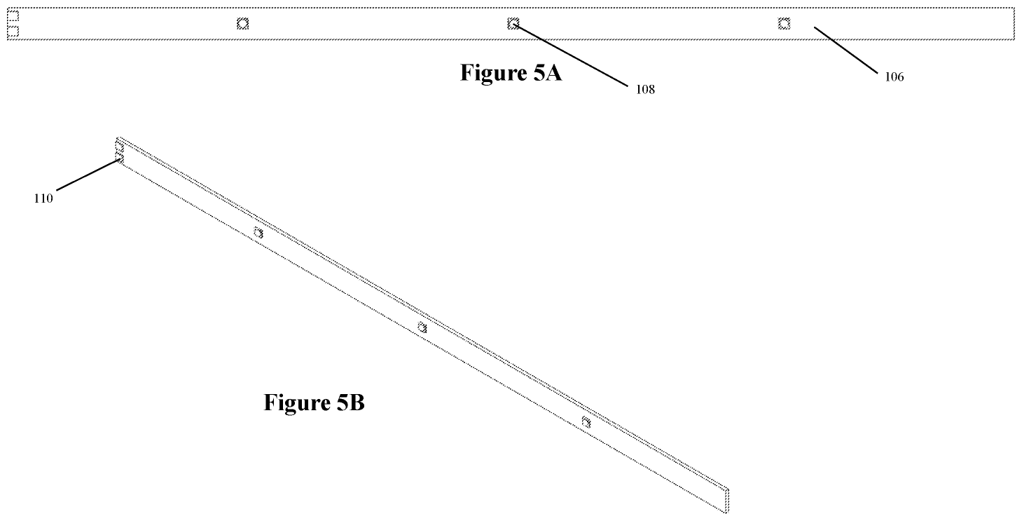

FIGS. 4A and 4B show example singular light modules 118. FIGS. 5A and 5B show example linear light modules 106. In some examples, the light modules may comprise one or more sets of positive and negative soldering pads for applying power. In some examples the circuits boards may comprise connectors or connecting points 110 for applying power. The light modules 106/118 may comprise at least one light emitter 108. In some examples, the light modules 106/118 may comprise one or more light emitters 108. The light modules may comprise design elements that allow for certain types of mounting, i.e., mounting holes 124 for the use of mounting screws. The light modules 106 may be linear as shown in FIGS. 5A and 5B.

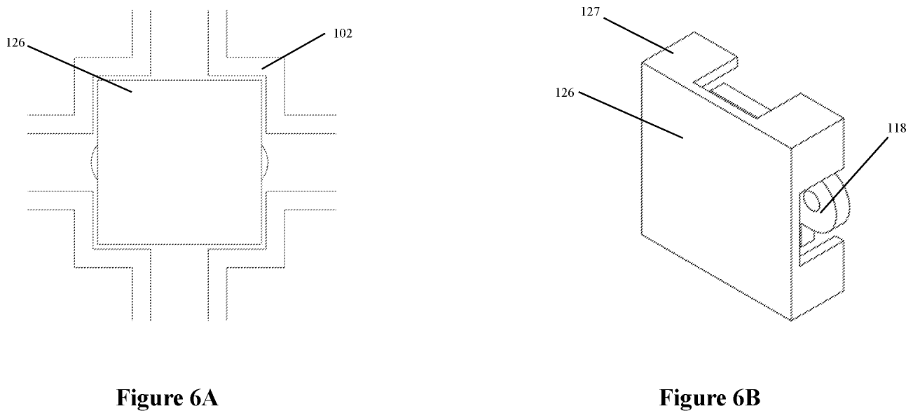

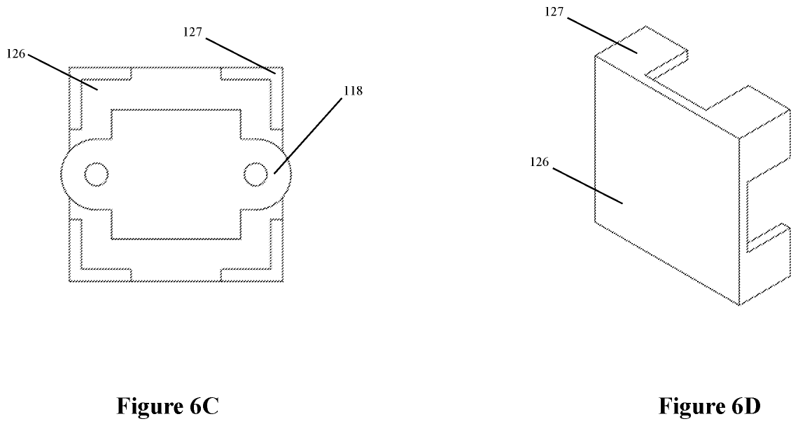

In some examples, a lens 126 may be placed into the frame over the light emitters. FIGS. 6A, 6B, 6C, and 6D show example lenses 126. The lens may be transparent or translucent. The lens may be placed over individual light emitters 108, in some examples. The lens material may be selected to have high transmission of disinfecting wavelength ranges. For example, the lens may allow for 75-100% transmission of wavelengths within the range of 380-420 nm. In some examples, the lens is fixed in place. In some examples, the lens rests in the frame and is held into place with an additional cover component. The lens 126 of FIGS. 6A-6D comprises a main section through which disinfecting light transmits through. Additionally, the lens 126 may comprise standoffs 127 that allow the lens to properly rest and/or fit into the frame. The standoffs may be a distance that allows for a specified gap between the light emitter(s) and the lens surface. The shape of lens 126 is an example only, other shapes and/or sizes are possible. The shape and/or size of the lens may depend on the form factor of light module 118.

In some examples, the lens material may comprise an antistatic element to prevent the build-up of particles on it. In some examples the lens may comprise an antistatic coating to prevent the build-up of particles on it.

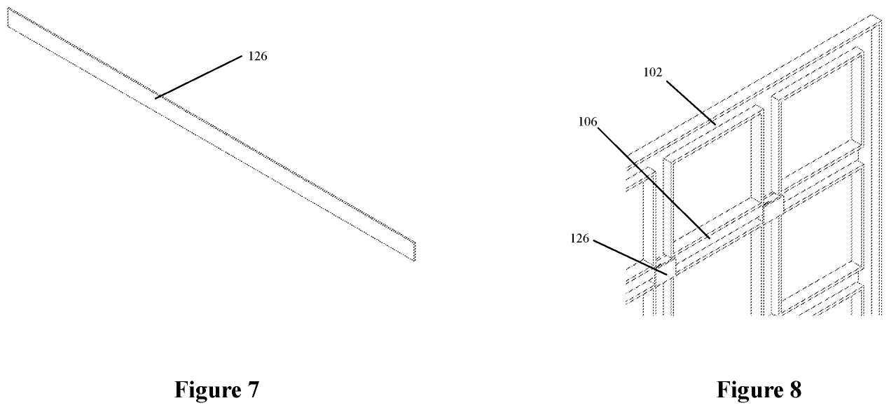

In some examples, as shown in FIG. 7, the lens 126 may be linear in shape, and placed over linear light modules 106 disposed with light emitters. In some examples shown in FIG. 8, individual lenses 126 may be used over the light emitters populated onto a linear light module 106.

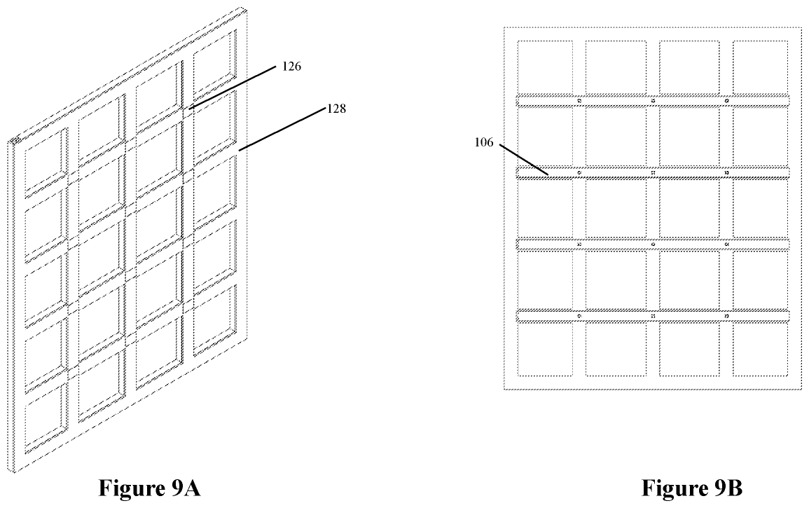

In some examples, there is a cover over the frame. The cover may hide wiring and light module areas not comprising light emitters. The cover may fit over the frame and be mechanically fastened and/or held in place with bendable tabs. The cover may comprise cut outs where the light emitters and/or lenses are to allow for the light to pass through. FIG. 9A shows an example where the cover 128 has cut outs over each individual light emitter for the placement of a lens 126 and/or to allow light to transmit through. FIG. 9B shoes an example where the cover 128 has full linear cutouts over a linear light module 106 to allow for the placement of a linear lens and/or allow light to transmit through.

In some examples, the cover material may comprise an antistatic element to prevent the buildup of particles on it. In some examples the cover may comprise an antistatic coating to prevent the buildup of particles on it.

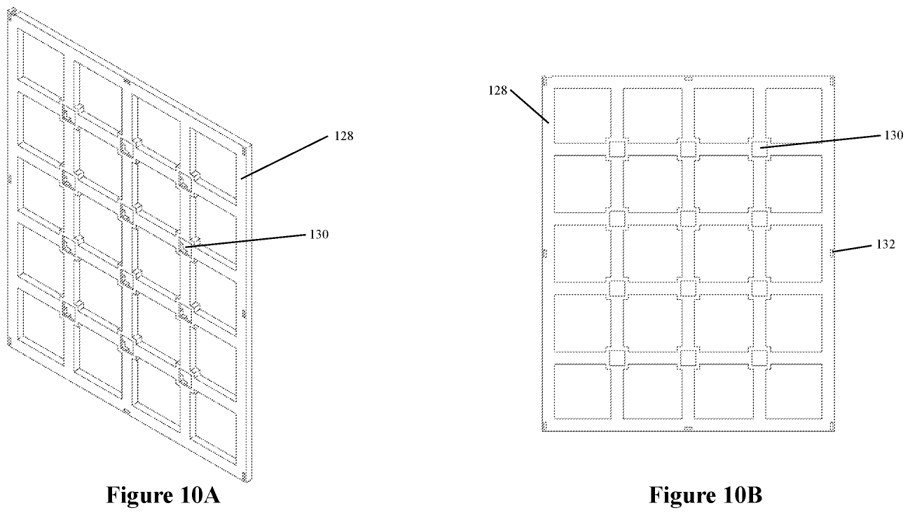

FIGS. 10A-10B show an example cover 128. Cover 128 has cutouts 130 at the central grid intersections to allow the light to pass through and/or for lens placement. The cover may hold the lenses in place. The cover may be formed such that it fits over the top of the frame. The cover may be formed such that it fits over the top of the frame and may press down such that the bottom of the cover is flush with the back of the frame.

In some examples, the standoffs are built into the cover. In some examples the standoffs are built into the frame. In examples where the standoffs are built into the frame, the cover may comprise cut outs to allow the standoffs to pass through the cover. FIG. 10B shows example cutouts 132 for allowing the standoffs from the frame to pass through.

The antimicrobial filter layer may comprise a frame, cover, one or more light emitters disposed onto one or more substrates, one or more lenses disposed over the light emitters, and associated power and connection components.

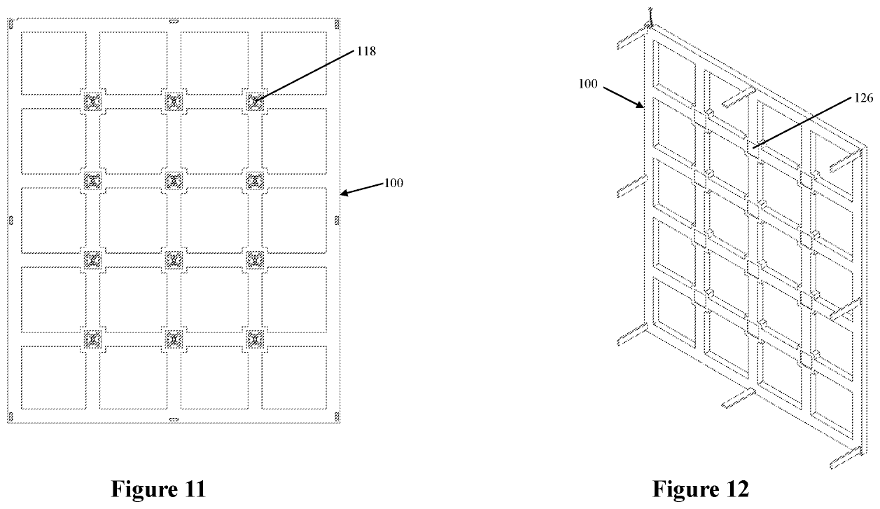

FIG. 11 shows an example of light modules 118 installed at the central grid intersections of an antimicrobial filter layer 100. FIG. 12 shows the lenses 126 installed at the central grid intersections over the light emitters of an antimicrobial filter layer 100.

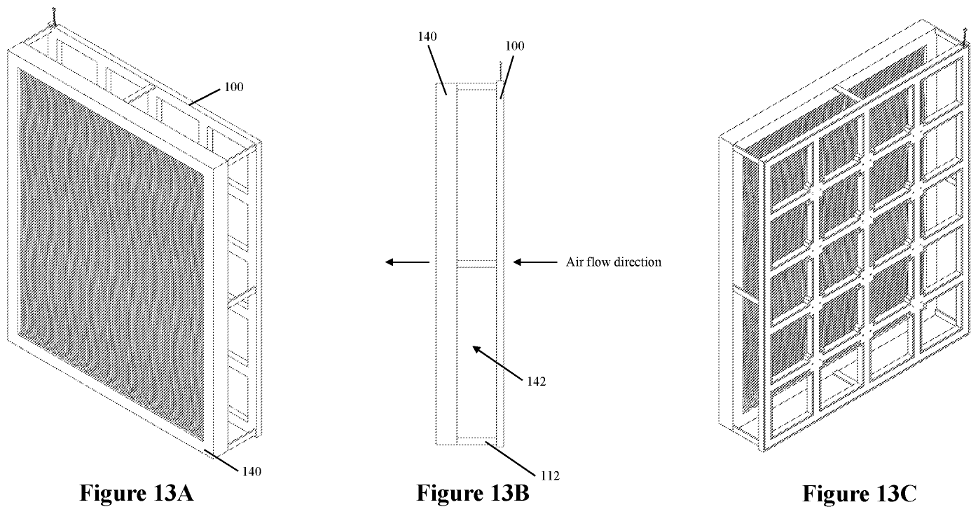

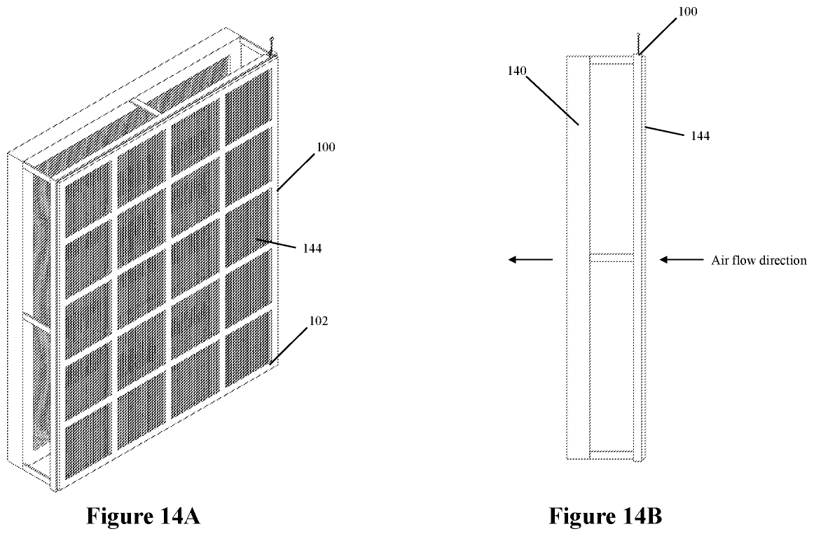

In some examples, the fibrous media filter rests directly against the standoffs from the antimicrobial filter layer. The fibrous media filter may be removable from the antimicrobial filter layer for cleaning and replacement. FIGS. 13A-B show the placement of a fibrous media filter 140 with the antimicrobial filter layer 100. The fibrous media filter 140 rests against the standoffs 112 of the antimicrobial filter layer 100 to create a gap 142 for light distribution from the light emitters. The air flows through the antimicrobial filter layer 100 into the fibrous media filter 140.

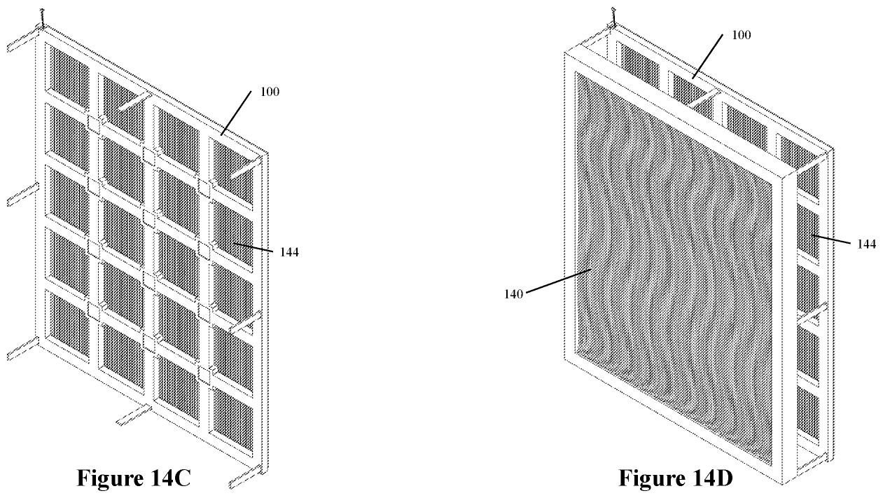

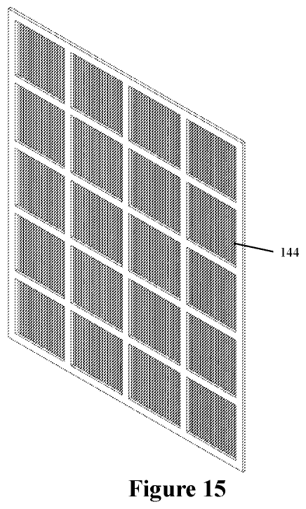

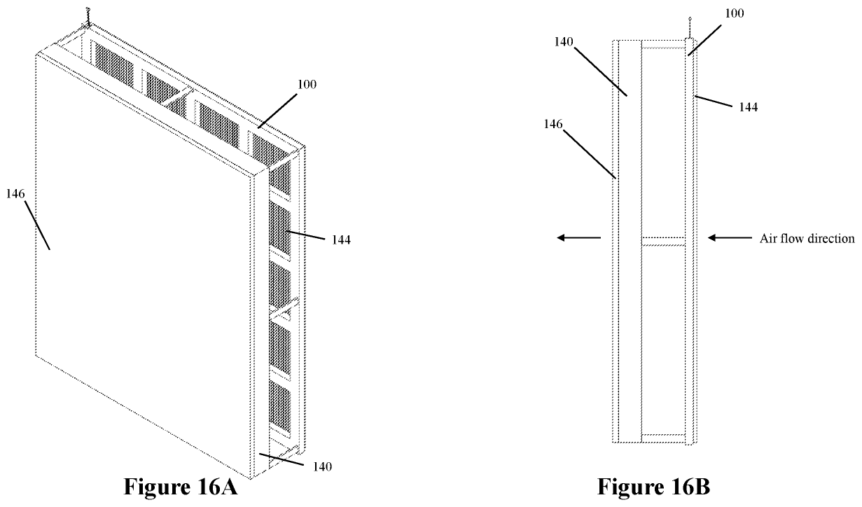

In some examples, the antimicrobial filter may comprise a pre-filter built into it or removably coupled to it. FIG. 14A shows an example pre-filter 144 coupled to the air intake side of the antimicrobial filter layer 100. In some examples, the pre-filter 144 is removably coupled to the antimicrobial filter layer 100. In some examples where the pre-filter 144 is removably coupled to the antimicrobial filter layer 100, magnets may be used for holding it in place. The pre-filter 144 may be built into the frame 102 of the antimicrobial filter layer 100.

In some examples, the air intake of the air purification device occurs on the opposite side of the light emitters. The light emitters may be facing the fibrous media filter. FIG. 14B shows air flow direction and FIGS. 14C-14D show an example air flow structure. The air may be directed through the device in the following order: pre-filter 144, antimicrobial filter layer 100, and then fibrous media filter 140.

FIG. 15 shows an example pre-filter 144 uncoupled or removed from the antimicrobial filter layer. The pre-filter 144 may be flexible or rigid. The pre-filter 144 may comprise specific pore sizes for blocking specific types of airborne particles. The pre-filter 144 may be easily removable for cleaning or replacement. The pre-filter may be made of an easily cleanable, and water resistant material.

In some examples, an adsorbent media filter may be removably coupled to the fibrous media filter. FIG. 16A shows an example orientation of the filter layers. The air may pass through the device in the following order: pre-filter 144, antimicrobial filter layer 100, fibrous media filter 140, and adsorbent media filter 146. FIG. 16B shows this example air flow structure from a side-perspective.

In some examples, there are no filter layers between the antimicrobial filter layer and the fibrous media filter to allow for direct light coverage of the fibrous media filter from the light emitters of the antimicrobial filter layer.

In some examples, the antimicrobial filter layer is a packaged device comprising the antimicrobial filter layer, a pre-filter, and an adsorbent media filter. The filter layers may be removably coupled together.

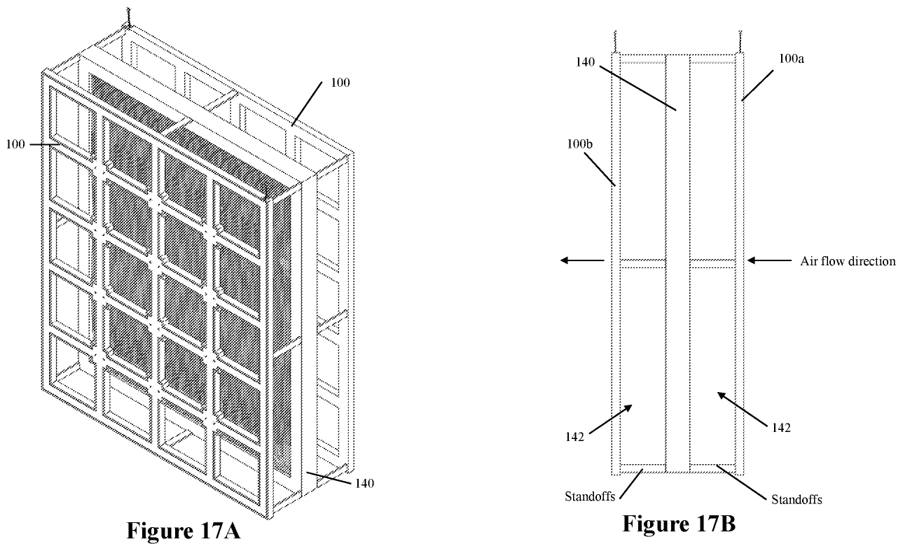

In some examples, there may be two antimicrobial filter layers or the antimicrobial filter layer may comprise surfaces on two or more sides of the fibrous media filter. FIG. 17A shows an example structure where there are two antimicrobial filter layers 100. In some examples using a flat fibrous media filter 140, the antimicrobial filter layers 100 are mirrored on each side of the fibrous media filter 140, providing disinfecting illumination to both sides of the fibrous media filter 140. In this example, the fibrous media filter 140 is removable within a clam shell antimicrobial filter layer 100 package. An example air flow order structure may be as follows: antimicrobial filter layer 100a, fibrous media filter 140, and antimicrobial filter layer 100b. FIG. 17B shows this example air flow structure. There are gaps 142 on each side of the fibrous media filter 140 to allow for light distribution onto the surface of the fibrous media filter 140.

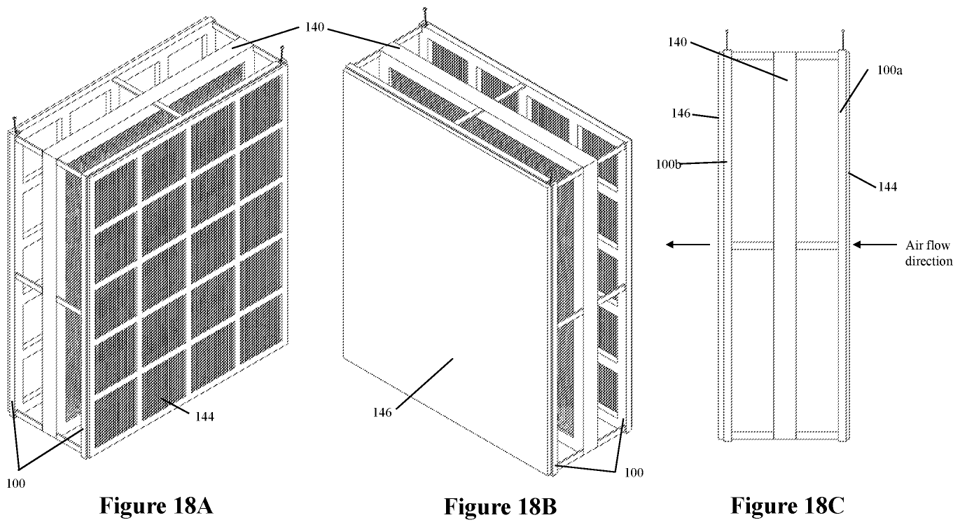

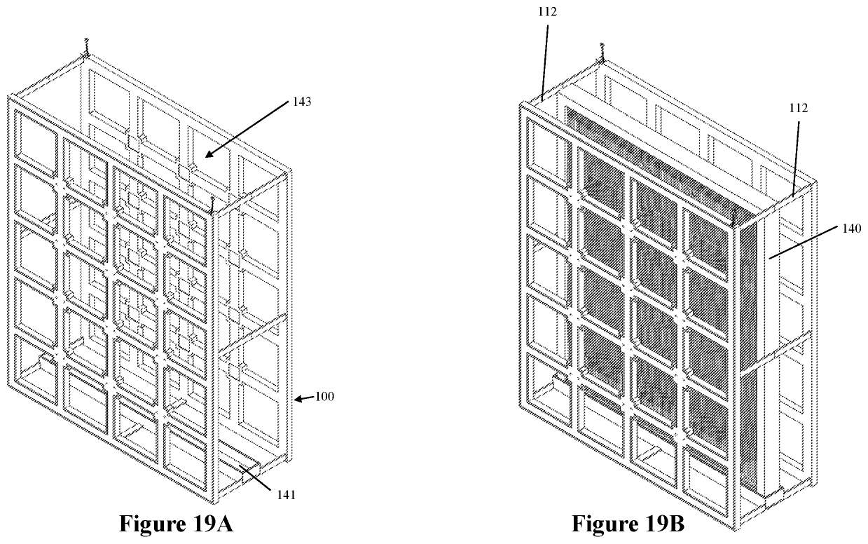

In some examples using multiple antimicrobial filter layers, a pre-filter may be integrated into or removably coupled to the air intake side of the antimicrobial filter layer. In some examples using multiple antimicrobial filters layers, an adsorbent media filter may be removably coupled to the air exit/output side of the antimicrobial filter layer. The orientation of the filter layers may be adjusted and any order of the aforementioned filter layers is possible. FIGS. 18A and 18B show an example structure using two antimicrobial filter layers 100, a pre-filter 144, and an adsorbent media filter 140. An example air flow structure shown in FIG. 18C may be as follows: pre-filter 144, antimicrobial filter layer 100a, fibrous media filter 140, antimicrobial filter layer 100b, and adsorbent media filter 146.

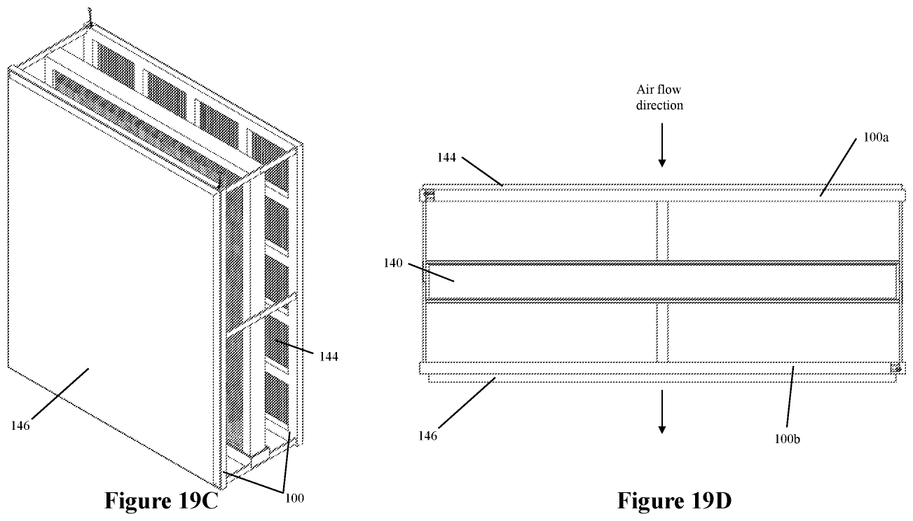

In some examples, the two sides of the antimicrobial filter layer may be combined into a single device which allows for a fibrous media filter to slide into the middle of it. FIGS. 19A and 19B show this example device. The bottom of the antimicrobial filter layer 100 which bridges the two layers together, comprises an area 141 for placing the fibrous media filter and holding it in place. Each side of the antimicrobial filter layer 100 comprises standoffs 112 for guiding the fibrous media filter 140 in place and holding it at the correct distance from the antimicrobial filter layer 100 such that proper illumination can occur on both surfaces of the fibrous media filter 140. In some examples, the single device may include a pre-filter 144 coupled to the air intake side and/or an adsorbent media filter 146 coupled to the air exit/output side. FIG. 19C shows this example structure. FIG. 19D shows an example air flow structure as follows: pre-filter 144, side one of the antimicrobial filter layer 100a, fibrous media filter 140 placed in the center of the antimicrobial filter layer, side two of the antimicrobial filter layer 100b, and adsorbent media filter 146.

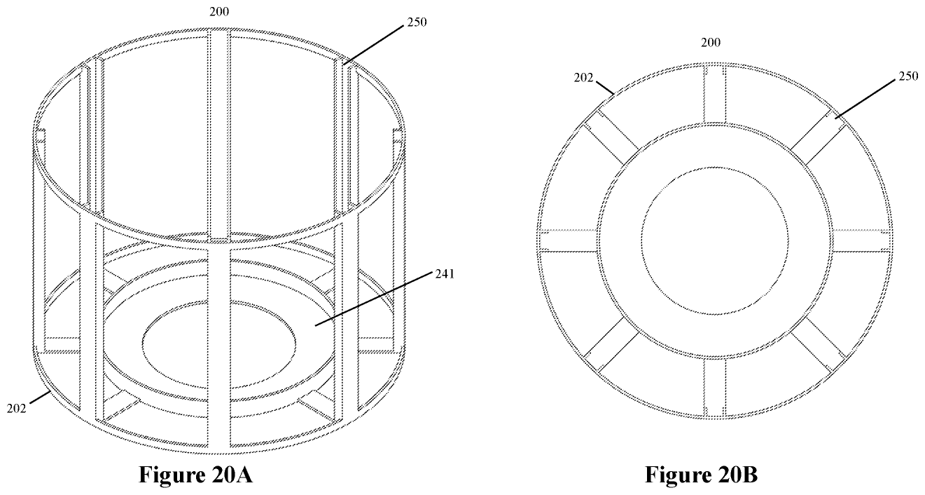

In some examples, the fibrous media filter may be cylindrical in shape. In examples where the fibrous media filter is cylindrical in shape, the antimicrobial filter layer may also be a cylindrical structure. An example cylindrical frame is shown in FIGS. 20A and 20B. The frame 202 of the antimicrobial filter layer 200 may comprise a base 241 with an area for the cylindrical fibrous media filter to rest in place at a distance from the light emitters. The frame 202 may additionally comprise channels 250 for mounting one or more light modules 206 disposed with one or more light emitters. The channels 250 may run parallel with the sides of the cylindrical fibrous media filter. In some examples, there are gaps between the channels 250 of the frame to allow air to pass through. In some examples there are one or more channels. In some examples there are at least 2, 4, 6, 8, 10, or 12 channels comprising light emitters 208. In some examples there are gaps at the base of the frame. In some examples the base of the frame is closed off.

In some examples, the cylindrical antimicrobial filter layer frame 202 is built into an air purification or HVAC device as part of its structure. The frame 202 and/or the base 241 may be part of the structure of an air purification or HVAC device. In some examples, the antimicrobial filter layer 200 may be separate from the air purification or HVAC device and may be removable or fixed into place within the air purification or HVAC device.

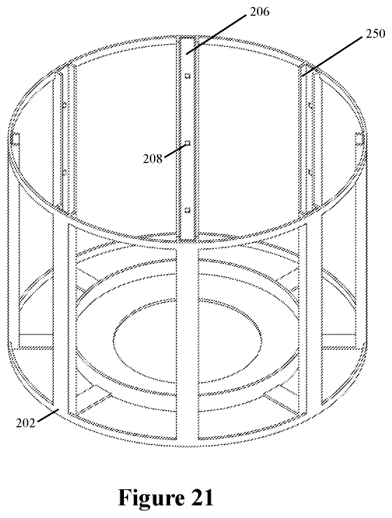

FIG. 21 shows an example cylindrical antimicrobial filter layer frame 202 with light modules 206 installed in the channels 250. The light modules 206 may be linear module populated with one or more light emitters 208. In some examples there may be 3 or 4 light emitters. In some examples there may be 4 or more light emitters.

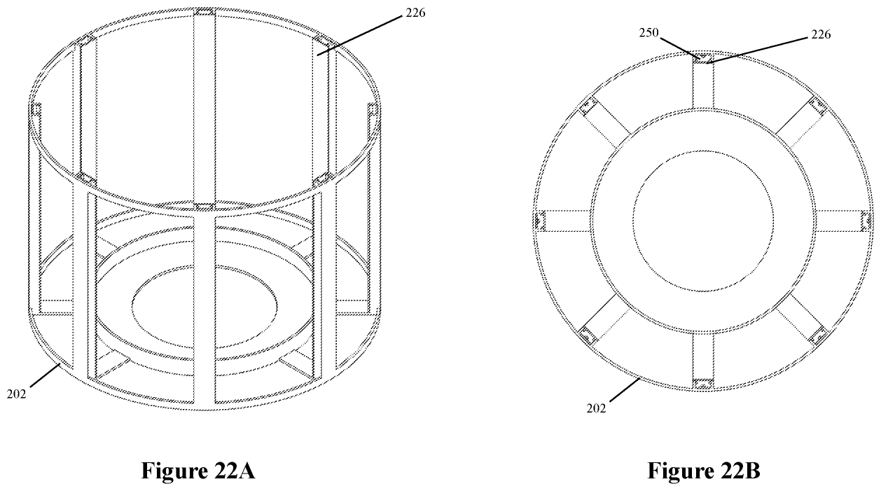

In some examples, and as described above, a lens may be disposed over the light module 206 and/or within and/or over the channels 250 of the frame 202. The lens may slide into the channel 250. There may be a lens covering each light module 206. FIGS. 22A and 22B show example lenses 226 installed within the frame 202.

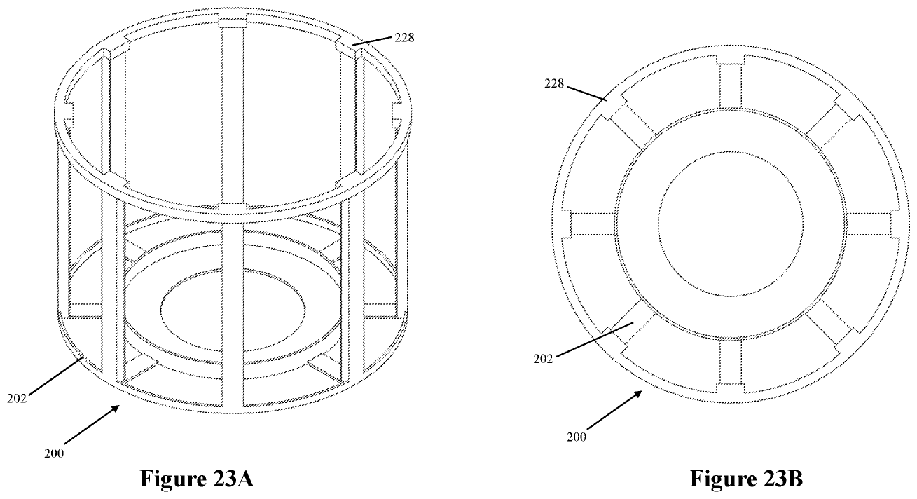

In some examples, a cover is placed over the cylindrical frame of the antimicrobial filter layer to hide the internal components and/or hold the lens in place. FIGS. 23A and 23B show an example cover 228 in place on the frame 202 of the cylindrical antimicrobial filter layer 200.

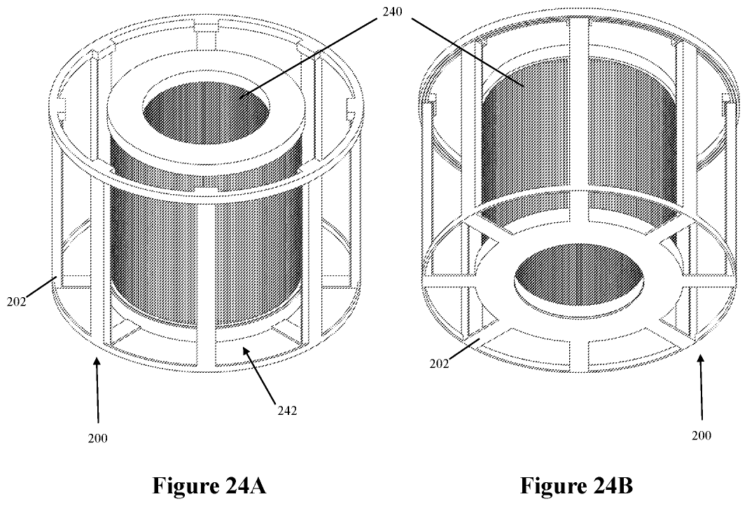

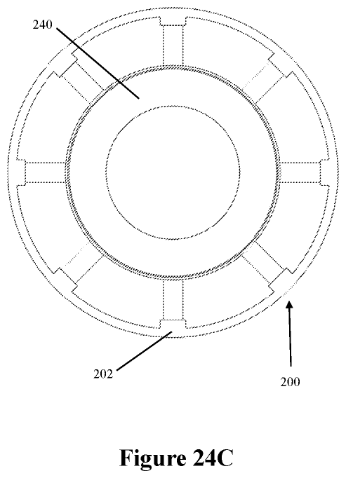

FIGS. 24A, 24B, and 24C show a cylindrical fibrous media filter 240 placed within the cylindrical antimicrobial filter layer 200. The frame 202 of the antimicrobial filter layer 200 may comprise a structure for the fibrous media filter 240 to be placed at a distance from the light emitters. The distance may be greater than or equal to 0.25 in. In some examples the distance may be less than or equal to 2 in. In some examples the distance may be less than or equal to 12 in. In still other examples, the distance may be, for example, at least, greater than, less than, equal to, or any number in between about 0.25 in, 0.5 in, 0.75 in, 1 in, 1.25 in, 1.5 in, 1.75 in, 2 in, 2.25 in, 2.5 in, 2.75 in, 3 in, 3.25 in, 3.5 in, 3.75 in, 4 in, 4.25 in, 4.5 in, 4.75 in, 5 in, 5.25 in, 5.5 in, 5.75 in, 6 in, 6.25 in, 6.5 in, 6.75 in, 7 in, 7.25 in, 7.5 in, 7.75 in, 8 in, 8.25 in, 8.5 in, 8.75 in, 9 in, 9.25 in, 9.5 in, 9.75 in, 10 in, 10.25 in, 10.5 in, 10.75 in, 11 in, 11.25 in, 11.5 in, 11.75 in, and 12 in.

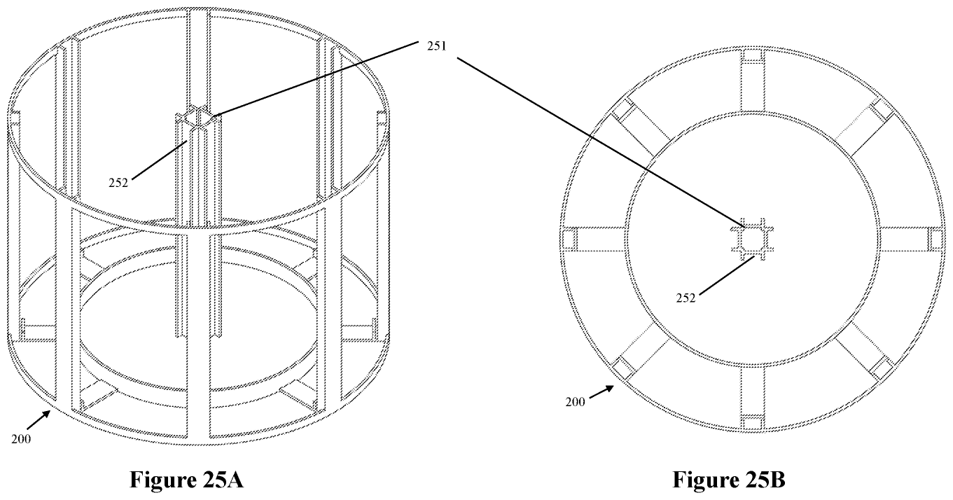

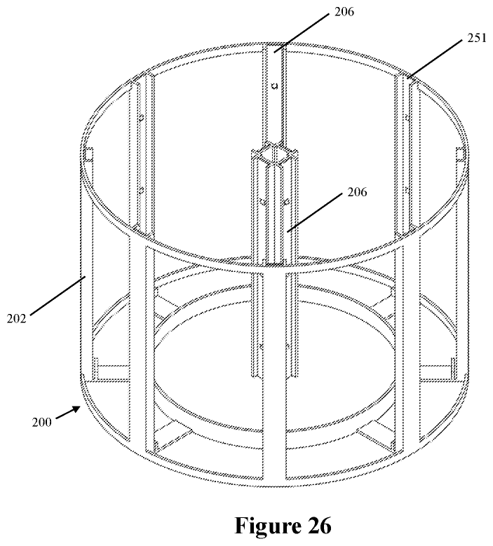

In some examples, an inner core element is built into the cylindrical antimicrobial filter layer to illuminate the inside surface of the cylindrical fibrous media filter. FIGS. 25A and 25B show the inner core 251 and inner core channels 252 built into the cylindrical antimicrobial filter layer 200. FIG. 26 shows example light modules installed into the channels of the antimicrobial filter layer comprising an inner core 251. In some examples, the inner core 251 may comprise four channels 252 for placing light modules. In some examples, the inner core 251 may comprise one, two or three channels 252. In some examples, the inner core 251 may comprise four or more channels 252 for placing light modules. FIG. 26 shows four light modules 206 placed in four channels 251 of the inner core 251 of the frame 202 of the cylindrical antimicrobial filter layer 200.

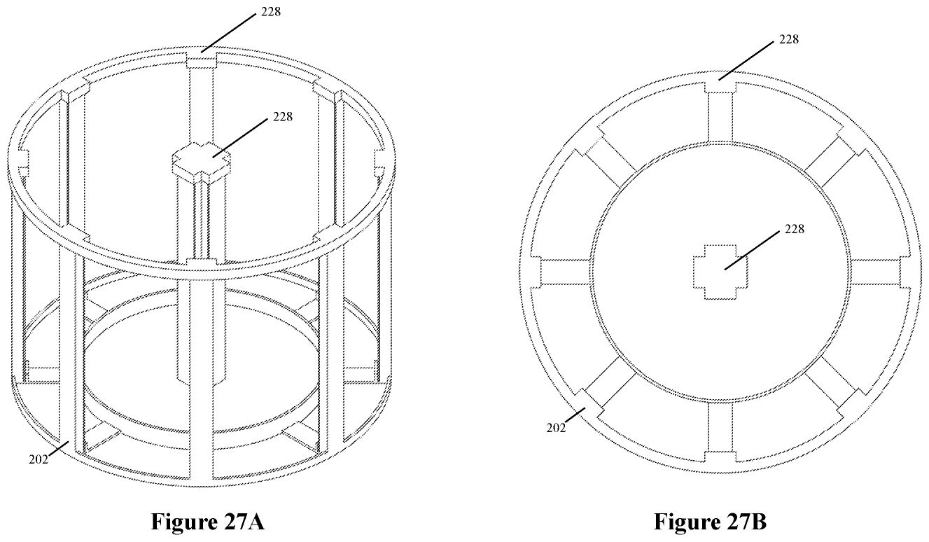

In some examples, an additional cover may be used over the core of the antimicrobial filter layer to hold the lenses in place and/or hide the internal components. The cover may be formed such that it fits down around the core. FIGS. 27A and 27B show this example cover 228 over the core area.

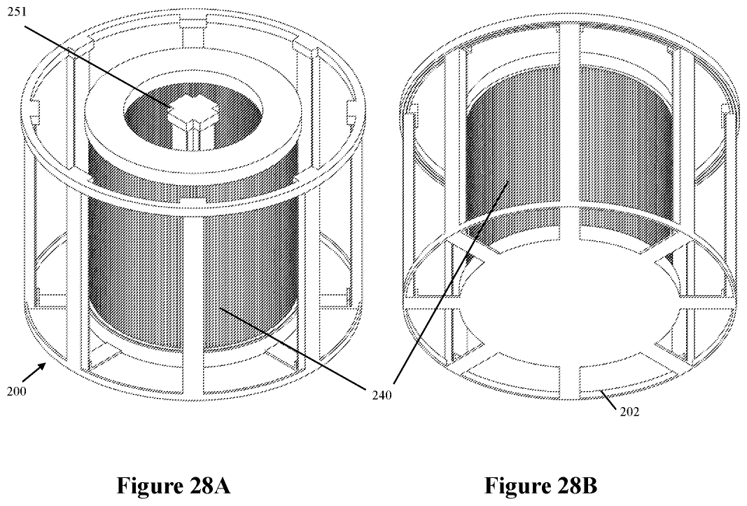

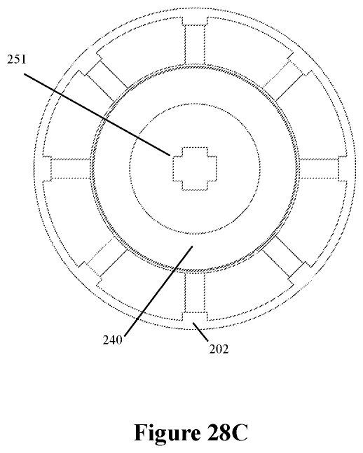

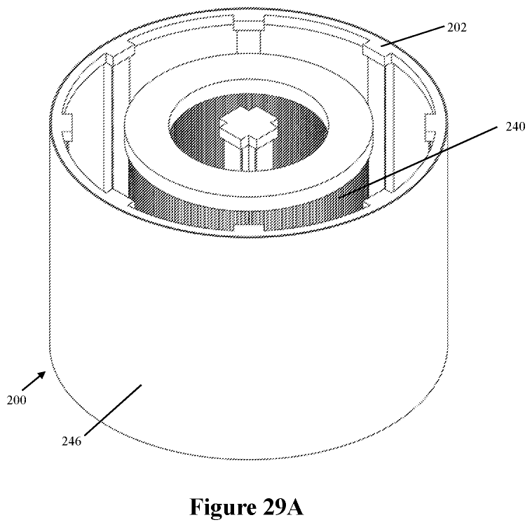

FIGS. 28A, 28B, and 28C show a cylindrical fibrous media filter 240 installed within the cylindrical antimicrobial filter layer 200 comprising an inner core 251.

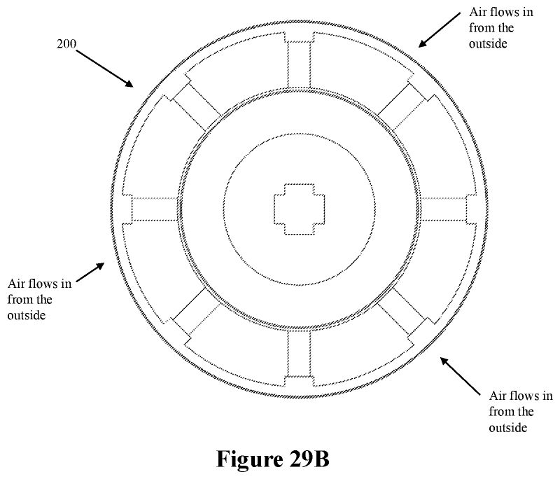

In some examples a flexible removable adsorbent media filter may be wrapped around the outside of the cylindrical antimicrobial filter layer. FIGS. 29A and 29B show an example adsorbent media filter 246 wrapped around the outside of the cylindrical antimicrobial filter layer 200. The adsorbent media filter 246 may be flexible or rigid and vary in thickness. In some examples, a removable pre-filter may be wrapped around the outside of the cylindrical antimicrobial filter layer 200 or built into the antimicrobial filter layer 200 as a single device.

In some examples the top of the cylindrical antimicrobial filter layer has an open ring shape for easily placing and removing a cylindrical fibrous media filter.

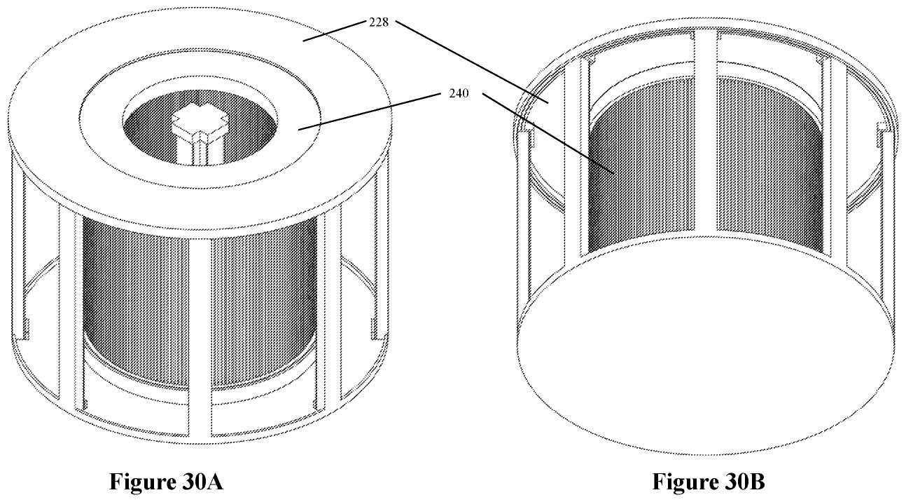

In some examples, the cylindrical filter layer comprises a cover that covers the bottom surface and/or the gap or open ring shape in the top surface of the antimicrobial filter layer such that the air is forced to pass through the outside of the fibrous media filter into the inside, and then exit/output out the top of the cylindrical fibrous media filter as shown in FIGS. 30A and 30B. In some cases, the cover is not required because the design forcing the air flow is built into the air purification or HVAC device the antimicrobial filter layer is installed into. The cover may be removable for placing the fibrous media filter into the antimicrobial filter layer and holding it in place. In some examples, the cover may not need to be removed to place the fibrous media filter in place. FIGS. 30A and 30B also show an example cover 228 used to control air flow.

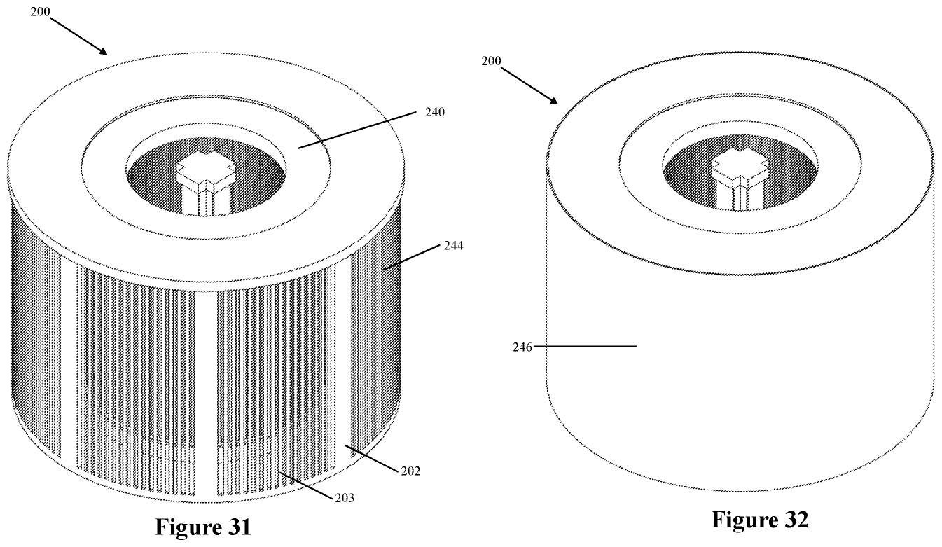

FIG. 31 shows an example antimicrobial filter layer 200 where the pre-filter 244 is built into the frame 202 of the antimicrobial filter layer 200. The antimicrobial filter layer 200 comprises linear elements 203 within the previous gaps at a spacing sufficient to black certain airborne particles such as dust, hair, etc. FIG. 32 shows an adsorbent media filter 246 wrapped around the outside of the antimicrobial filter layer 200 which comprises a built-in pre-filter.

In some examples, a fan is used to pull the air through the filter layers comprising the antimicrobial filter layer. The fan may be built into the air purification device or the HVAC system the antimicrobial filter layer is being utilized in.

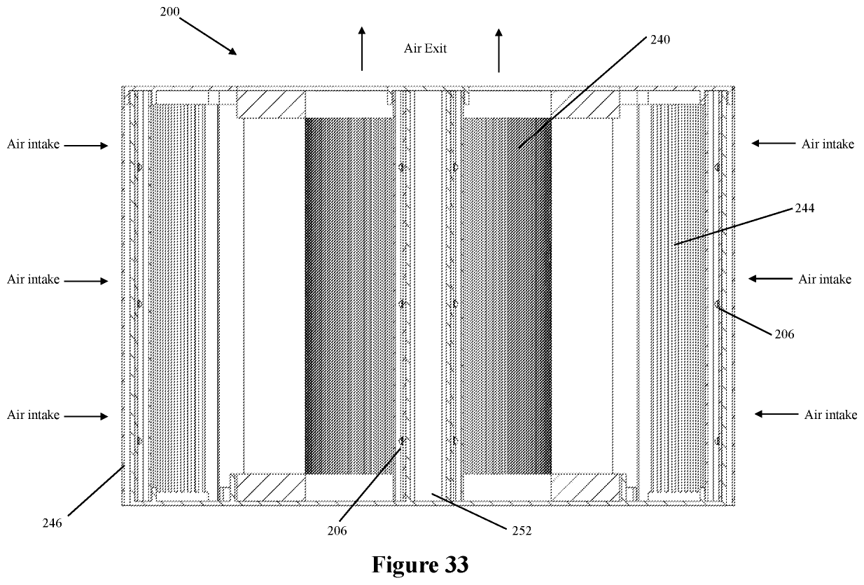

FIG. 33 shows a cross-sectional view of FIG. 32 and shows an example air flow structure. The air may flow through the external adsorbent media filter 246, through the prefilter gaps built into the antimicrobial filter layer 200, through the cylindrical fibrous media filter 240 and into the center of the fibrous media filter where it then exits out the top center. The exit point may vary in location and other locations are possible. The air may exit out the bottom of the device in some examples.

In some examples, the air may exit the air purification or HVAC device back into the same environment the air was collected from. In some examples, the air may exit the air purification or HVAC device through an exhaust that transfers the air to a different environment from which the air was collected from. In some examples the air may exit a section of the air purification or HVAC device into a different section of the air purification or HVAC device.

In some examples, surfaces designed for forcing air flow may be built into the antimicrobial filter layer. In some examples, surfaces designed for forcing air flow are built into the air purification or HVAC device the antimicrobial filter layer is installed into.

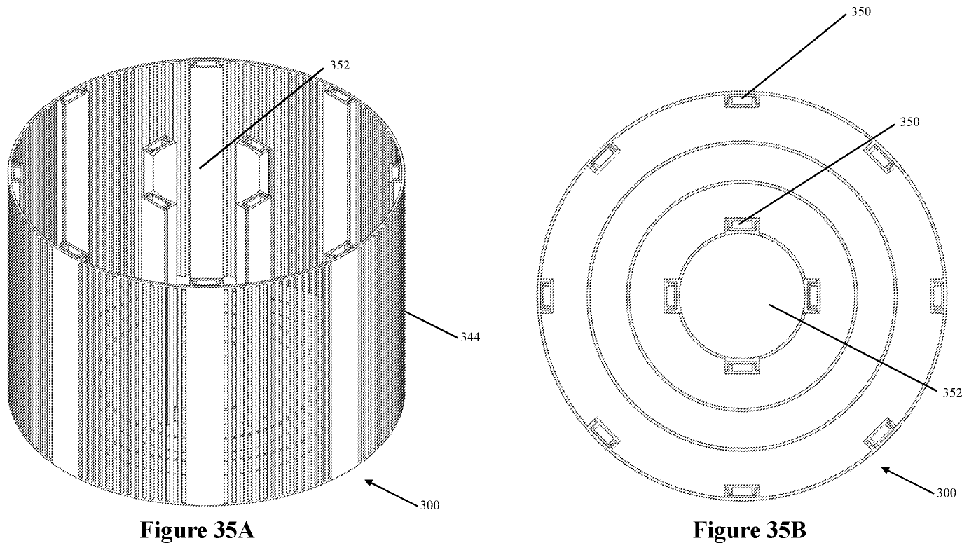

In some examples, a cylindrical antimicrobial filter layer may comprise a larger core for directing air flow through the center core and through an adsorbent media filter before exiting out the top or bottom. FIGS. 35A and 35B show an example cylindrical antimicrobial filter layer 300 with a larger core area 352 for placing an adsorbent media filter. The example antimicrobial filter layer may have a pre-filter 344 built into the frame. The example antimicrobial filter layer may include an area built into the bottom of the frame for the cylindrical fibrous media filter to be placed. The example antimicrobial filter layer may comprise channels for mounting light modules on the outer frame embedded within the pre-filter. Additionally, channels 350 may be located within the inner core, but spaced such that there is an interior gap within the inner core for the placement of an adsorbent media filter and for air flow.

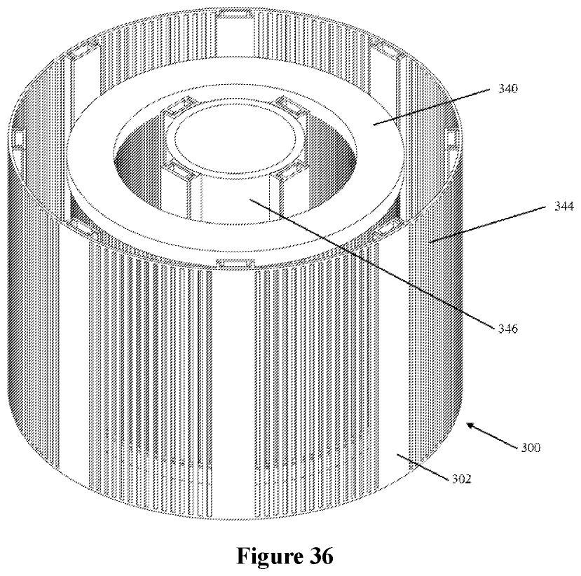

FIG. 36 shows the addition of the fibrous media filter 340 and the adsorbent media filter 346 into the structure of FIGS. 35A and 35B. The adsorbent media filter 346 may be cylindrical in shape. In some examples, the adsorbent media filter 346 may be a flat sheet that wraps around the interior of the central core. The central core has gaps that allow for the air to flow through the adsorbent media filter 346 and into the center of the device. The air may then exit out the top or bottom of the central cylinder.

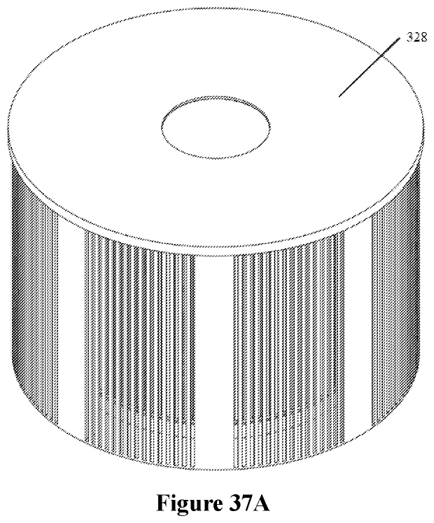

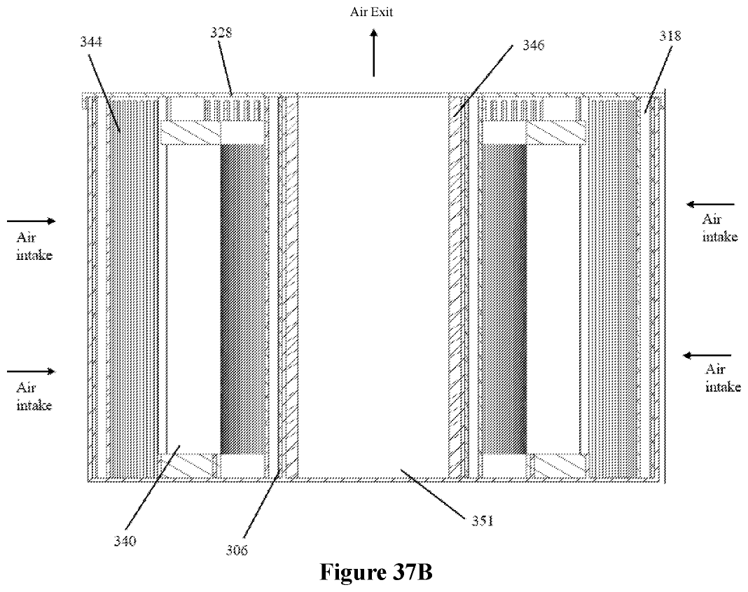

FIG. 37A shows an example cover 328 placed over the structure of FIG. 36 to control air flow and force air to the center of the device before exiting out the top or bottom.

FIG. 37B shows a cross-sectional view of FIG. 37A. FIG. 37B shows an example air flow structure. Air may first pass through the pre-filter 344 built into the antimicrobial filter layer frame, then through the gap, through the fibrous media filter 340, into the center core area where the air then passes through the center core 351 of the antimicrobial filter layer and through an adsorbent media filter 346. Once the air reached the center of the adsorbent media filter, it exits up or down out of the filter structure.

In some examples, the antimicrobial filter layer may be built into the air purification device. In some examples, the antimicrobial filter layer may rest inside the air purification device uncoupled. In some examples, the antimicrobial filter layer may be removably coupled to the inside of the air purification device.

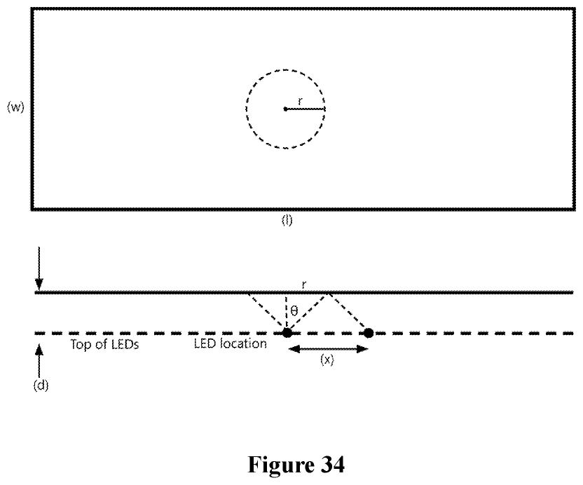

In some examples, the light emitter(s) may emit disinfecting light. The intensity of the disinfecting light from light emitter(s) may vary based on the angle the disinfecting light is emitted from the light emitter(s). FIG. 34 shows an example diagram of this concept. In some examples, disinfecting lighting element may have a beam angle of up to 180 degrees. In some examples, the beam angle may be 60, 120, and/or 130 degrees. The intensity of the disinfecting light may be highest in the center of a beam of disinfecting light emitted from the light emitter(s). In some examples, the intensity may be lower towards the edge of the beam of disinfecting light than the center of the beam. In some examples, the intensity at the edge of a beam of disinfecting light may be 50% of the maximum intensity which may occur in the center of the beam. In some examples, the intensity of the disinfecting light may decrease further from the light emitter(s). The disinfecting light may, for example, have a maximum intensity close to the light emitter(s) and the intensity may decrease as the disinfecting light travels further from the light emitter(s). Due to this, the light emitter(s) may be placed such that there is sufficient light coverage on the target surface, i.e., filter. The spacing of the light emitter(s) is based upon the distance between the light emitter(s) and the target surface, the radiometric power output of the light emitter(s), and the beam angle of the light emitter. In some examples, the light emitter(s) create a circular light coverage area on the target surface. In some examples, the light emitter(s) will be positioned such that the contour line receiving 50% of the maximum intensity which may occur in the center of the beam on the target surface provided from one light emitter, overlaps with the contour line receiving 50% of the maximum intensity which may occur in the center of the beam on the target surface provided from a separate light emitter such that the areas on the target surface receiving less than 50% of the maximum intensity which may occur in the center of the beam is minimized. In some examples, the overlap between the emitters may be, for example, at least, greater than, less than, equal to, or any number in between about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79% and 80% of the maximum intensity.

In some examples, a surface to be disinfected may be in close proximity to a light emitter. In such examples, a device may require more light emitters than would otherwise be necessary for disinfection. The area illuminated by a single light emitter may be limited by a beam angle of the light emitter. The same light emitter may illuminate a larger surface area of the surface to be disinfected if the light emitter is moved further away. Therefore, the device disclosed may need an increased number of light emitters to cover the entire surface area of the surface to be disinfected with disinfecting light, as compared to a further distance. FIG. 34 illustrates angles of light emitted from light emitters disclosed herein. Light emitters may be spaced a distance from the surface to be disinfected. The light emitters may emit a light that spreads outwardly toward the surface at a beam angle. The beam angle may comprise half of an angle of light emitted from the light emitter, in degrees, where the intensity of light is at least 50% of light emitter's maximum emission intensity. In some examples, the light emitter may comprise LEDs and the beam angle may be 130 degrees, e.g., the angle of light emitted from the light emitter where the intensity of light is at least 50% of the maximum emission intensity is 130 degrees. In some examples where light from the light emitter does not possess rational symmetry, the beam angle may be given for two planes at 90 degrees to each other.

A total surface area illuminated by one light emitter, as shown in FIG. 34, may be determined by the beam angle and the distance from the light emitter to the surface intended to be disinfected. A light emitter with a larger beam angle may provide a larger total surface area illuminated by one light emitter. An increased distance between the light emitter and the surface may also increase the total surface area illuminated by one light emitter. The total number of light emitters that may be needed to disinfect the entire surface to be disinfected may be based on the total surface area illuminated by one light emitter. As the distance from the surface intended to be disinfected to the light emitter decreases, the number of light emitters that may be needed to disinfect the surface may increase.

In some examples, the media of the fibrous media filter may allow for wavelengths of light within the range of 380-420 nm to transmit through the media and pleats/structure of the fibrous media filter. This media may be light in color, for example, to allow transmission.

In some examples, a control system may be operatively coupled to the antimicrobial filter layer and/or the air purification device or system it is utilized in. The example control system may be operative to control operational features of the device such as but not limited to: a duration of illumination, type of light emitter used, exiting light color, light intensity, and/or light irradiance. The control system may include any now known or later developed processor, microcontroller, system on a chip, computer, server, network device, mesh network device, internet-of-things device, mobile device, etc. The light device may also include at least one sensor coupled to control system to provide feedback to control system. In some examples, sensor(s) may sense any parameter of the control environment of the device, motion of a user, motion of structure to which device is coupled, temperature, humidity, light reception, position of panels covering the antimicrobial filter layer, opacity of the fibrous media filter, presence and/or level of volatile organic chemicals, air quality and/or air particulates and/or presence of microorganisms on exterior surface, combinations thereof, etc. Sensor(s) may include any now known or later developed sensing devices for the desired parameter(s). The control system with sensor(s) (and without) can control operation to be continuous or intermittent based on external stimulus, and depending on the application.

In some examples, the control system and/or lights may be wired or wirelessly coupled to the internet (with or without a gateway) and a cloud or on-premises server to control or record data associated with the control system and/or light emitters. In some examples, usage patterns and determinations regarding time-on in different modes, irradiance or dosage thresholds being met may be recorded.

Some microorganisms may respond differently to different wavelengths. In some examples, the control system may adjust the spectrum of the light based on the type of microorganism. For instance, some microorganisms may require high levels of 405 nm light, e.g., >1 mW/cm² for several hours. In some examples, the 405 nm light may be required, for example, at least, greater than, less than, equal to, or any number in between about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71 and 72 hours. The same microorganisms may only require 10 uW/cm² at 222 nm, for example, in a smaller time period (minutes) to achieve the same kill. Therefore, it may be beneficial to know the type of microorganism so that the spectrum can be tailored to it. In some examples, the control system may be pre-programed to target specific microorganisms. In some examples, data regarding dosage, irradiance, etc. for a specific microorganism may be input manually. In some examples, the control system or remote server may comprise a database containing optimal spectra for different types of microorganisms. In some examples, a bioburden sensor may be used to detect the type of microorganism and transmit information to the control system for targeting the microorganism. In some examples, the bioburden sensor may be an autofluorescence sensor, which may comprise a light emitter to cause excitation of the bioburden, and a sensor to measure the resulting emission from the bioburden. This bioburden sensor may interact with the control system or remote database to cause tuning of the light's spectrum.

A computing device (e.g., a controller) may be comprised by the device disclosure and may perform the functions of various control systems described herein, and/or any other computer, controller, or processor-based function described herein. The computing device may implement, for example, a control system for control of various lighting parameters, as described herein. In some examples, the computing device, in communication with one or more sensors and one or more lighting devices may implement lighting controls based on sensor measurements. In some examples, the computing device may be a microcontroller configured to implement the functions of various control systems described herein.

The computing device may include one or more processors, which may execute instructions of a computer program to perform any of the features described herein. The instructions may be stored in any type of tangible computer-readable medium or memory, to configure the operation of the processor. As used herein, the term tangible computer-readable storage medium is expressly defined to include storage devices or storage discs and to exclude transmission media and propagating signals. For example, instructions may be stored in a read-only memory (ROM), random access memory (RAM), removable media, such as a Universal Serial Bus (USB) drive, compact disk (CD) or digital versatile disk (DVD), floppy disk drive, or any other desired electronic storage medium. Instructions may also be stored in an attached (or internal) hard drive. The computing device may include one or more input/output devices, such as one or more sensors, lighting devices, display, touch screen, keyboard, mouse, microphone, software user interface, etc. The computing device may include one or more device controllers such as a video processor, keyboard controller, etc. The computing device may also include one or more network interfaces, such as input/output circuits (such as a network card) to communicate with a network such as example network. The network interface may be a wired interface, wireless interface, or a combination thereof. The computing device may comprise one or more timers to measure time. One or more of the elements described above may be removed, rearranged, or supplemented without departing from the scope of the present disclosure.

Various methods, devices, and systems described herein may use a control system to implement various lighting controls in the device disclosed. The control system may be used to control/adjust various aspects of disinfecting light (e.g., dosage, radiant flux, color, time, wavelength, intensity, and/or irradiance). In various examples, the control system may be used to control similar parameters corresponding to other wavelengths of light as well. The other wavelengths of light may correspond to white light, ultraviolet (UV) light, and/or other wavelengths that are not configured for disinfection. In other examples, controls may be implemented to turn off the disinfecting light when an individual opens the device to change or check the various filter(s) or filter layer(s) disclosed herein.

The control system may comprise the use of sensors. The sensor(s) may comprise, for example, one or more of irradiance sensors, radiant intensity sensors, motion sensors, voice sensors, odor sensors, capacitive touch sensors, magnetic proximity sensors, light sensors, infrared sensors, cameras, ultrasonic sensors, weight sensors, limit switches, and/or any other sensors.

The control system may comprise a timer. The timer may, for example, measure how long disinfecting light has been emitted towards an object. In some examples, the timer may measure the length of time since an enclosure was opened/closed. In some examples, enclosures using a timer to turn off the disinfecting lighting when a dosage has been met may also contain indication lighting to make the user aware that the disinfection cycle is complete. In some examples the indication light may be provided by additional lighting elements emitting colors outside of the disinfecting wavelength range, such as green light within the range of 520 to 560 nanometers.

In some examples, a module capable of emitting ultraviolet light may be used as a subcomponent within a device. The module may comprise of one of more of the following: LED PCBA, emitter, emitter package, driver or ballast, control circuitry, safety sensors, lens, reflector, cover, or enclosure. An LED PCBA may be a printed circuit board with surface mount LEDs. The module may also include driving circuitry, for example, to regulate current and voltage going to the LEDs. An emitter may be a UV emission source that is not an LED. A safety sensor may be used to prevent accidental exposure to the UV light. The safety sensor may comprise of an occupancy sensor, a timer, a button, or a control signal from a remote sensor or control system. The module may be enclosed such that UV light does not leak out and is only emitted through the lens.

Light emitters producing ultraviolet or visible light may comprise, for example, an LED, an array of LEDs, a laser, an array of lasers, a vertical cavity surface emitting laser (VCSEL), or an array of VCSELs. Other light emitters that may be used may include, for example, any emitter capable of emitting ultraviolet light including LEDs, fluorescent lamps without phosphor coatings, xenon arc lamps, mercury vapor, short-wave UV lamps made with fused quartz, black lights (fluorescent lamp coated with UVA emitting phosphor), amalgam lamps, natural or filtered sunlight, incandescent lamps with coatings that absorb visible light, gas-discharge (argon, deuterium, xenon, mercury-xenon, metal-halide, arc lamps, planar microcavity microplasma), halogen lamps with fused quartz, solid-state lamps, excimer lamps (such as Krypton Chlorine), etc. In some examples, an LED emitter may comprise at least one semiconductor die and/or at least one semiconductor die packaged in combination with light converting materials. In some examples, the light emitter may be fitted with optical components that may alter the path of the light (e.g., focus the light into a beam).

In some examples, the light emitter(s) may be populated onto a light module or substrate, i.e., circuit board module or printed circuit board. The light modules may vary in material, shape, size, thickness, flexibility, and otherwise be conformed to specific applications. Base material of the substrate may comprise a variety of materials such as, for example, aluminum, FR-4 (glass-reinforced epoxy laminate material), Teflon, polyimide, or copper.

In some examples, a light emitter or a light module may comprise a conformal coating. The conformal coating may comprise a polymeric film contoured to the light emitting subcomponent. The conformal coatings may provide ingress protection from, for example, condensation or other liquids.

In some examples a transparent or translucent surface may be required as part of the device as a lens or protective material layer. The transparent or translucent surface may allow for 50%-100% transmission of the disinfecting wavelengths. In some examples the materials incident to the disinfecting wavelength selected for the device may have high reflectance of the disinfecting wavelengths in order to increase the intensity/irradiance. The materials may be, for example, matte or glossy white plastics, or materials with mirror like finishes. In some examples, the transparent or translucent surface may allow for 70%-100% relative transmission of the disinfecting wavelengths to the overall visible spectrum wavelengths. In some examples, the transparent or translucent surface may allow for 50%-100% transmission relative to air of the disinfecting wavelengths. In some examples, materials that exhibit fluorescence under disinfecting light are not used due to the reduction in efficacy from absorption of disinfecting wavelengths and emission of longer wavelengths potentially out of the disinfecting wavelength range. In some examples, additives are added to the material to reduce gradual transmission reduction over time due to exposure to high temperatures.

In some examples, it may be desirable to dissipate heat generated by lighting elements or other components of a light emitter as disclosed herein. A decreased operating temperature may increase reliability and lifetime of a device. Heat may affect the peak wavelength and spectrum emitted by the light emitter(s). For example, as temperatures rise, peak wavelengths may shift to longer wavelengths. Similarly, as temperatures decrease, peak wavelengths may shift to shorter wavelengths. Therefore, it may be desirable to constrain the temperature to a certain range in order to maintain a desired peak wavelength or spectrum within some tolerance. In some examples, the light emitter or light module may be coupled to a heatsink. The heatsink may be made out of plastics, ceramics, or metals including, for example, aluminum, steel, or copper. The heatsink may also be made out of a plastic or ceramic material. In some examples the heatsink may be permanently coupled to a light emitter or light module, or otherwise considered a part of the assembly that makes up the light emitter or light module. In some examples the heat sink may be built into the structure the light module is mounted to, such as the frame of the antimicrobial filter layer.

The device disclosed herein may be powered through power outlets, electrical power supplies, batteries or rechargeable batteries mounted in proximity to the appliance, and/or wireless or inductive charging. Where rechargeable batteries are employed, they may be recharged, for example, using AC power or solar panels (not shown), where sufficient sunlight may be available. In some examples, AC power and an AC to DC converter, i.e. LED driver or power supply, may be utilized. In some examples, direct DC power may be utilized when available. In some example, the device will take in direct DC power from the device it is installed into, an air purifier for example.

In various examples described herein, light at a specified wavelength or wavelength range may correspond to light which has a maximum emitted energy/power/energy spectral density/power spectral density approximately at the specified wavelength or within the specified wavelength range, with reasonable variations (e.g., ±5 nm, ±10 nm, etc.).

The above discussed embodiments are simply examples, and modifications may be made as desired for different implementations. For example, steps and/or components may be subdivided, combined, rearranged, removed, and/or augmented; performed on a single device or a plurality of devices; performed in parallel, in series; or any combination thereof. Additional features may be added.

In some examples, a photocatalyst is an additional layer or element of the device, apparatus, or system.

In some examples, a photocatalyst is not used in the device, apparatus, or system.

In some examples, the light emitters emit light in the ultraviolet wavelength range.

In some examples, the light emitters emit light with a peak wavelength in the ultraviolet wavelength range.

In some examples, the light emitters do not emit light in the ultraviolet wavelength range.

In some examples, the light emitters emit light with a peak wavelength that is not in the ultraviolet wavelength range.

In some examples, the device is configured such that air can pass through gaps in the antimicrobial filter layer.

In some examples, the highest intensity of emitted light from the light emitter is emitted perpendicular to the fibrous media filter surface.

In some examples, the light emitters are LEDs.

In some examples, the fibrous media filter is removably attached to the antimicrobial filter layer.

In some examples, the fibrous media filter is not mechanically fastened to or adhered to the light emitters.

In some examples, the antimicrobial filter layer may comprise one or more light emitters.

In some examples, the antimicrobial filter layer may comprise two or more light emitters.

In some examples, the antimicrobial filter layer may comprise 12 or more light emitters.

In some examples the antimicrobial filter layer may comprise 24 or more light emitters.

In some examples, the antimicrobial filter layer is directed to disinfect the surfaces of a fibrous media filter.

In some examples, the antimicrobial filter layer is directed to disinfect the air passing through the antimicrobial filter layer.

In some examples, the antimicrobial filter layer is part of a system configured to allow airflow to pass through from an entrance point to an exit point.

In some examples, the antimicrobial filter layer may be used in a water purification process.

In some examples, the antimicrobial filter layer may be used in an air purification process.

In some examples, the antimicrobial filter layer is configured to disinfect a fibrous media filter.

In some examples, the system comprising the antimicrobial filter layer may additionally comprise a fibrous media filter.

In some examples, the system comprising the antimicrobial filter layer may additionally comprise a fibrous media filter and a pre-filter.

In some examples, the system comprising the antimicrobial filter layer may additionally comprise a fibrous media filter, a pre-filter, and an adsorbent media filter.

In some examples, the antimicrobial filter layer may be configured for flat shaped fibrous media filters.

In some examples, the fibrous media filter may be rectangular in shape, with a depth less than the length and width of the rectangular. This volumetric shape may be referred to as a flat shaped fibrous media filter or a flat rectangular fibrous media filter. This volumetric shape may be referred to as a three-dimensional flat rectangle.

In some examples, the antimicrobial filter layer may be configured for cylindrical shapes fibrous media filters.

In some examples, the antimicrobial filter layer may be a three-dimensional cylindrical shape with a hollow center.

In some examples, the air flow passes through perpendicular to the antimicrobial filter layer and fibrous media filter.

In some examples, there is a gap or distance between the antimicrobial filter layer comprising light emitters and the fibrous media filter.

In some examples, the gap or distance is 2 inches or less.

In some examples, the gap or distance is 2 inches or more.

In some examples, the gap or distance is less than 6 inches.

In some examples, the gap or distance is less than 12 inches.

In some examples, the gap is at least 0.25 inches.

In some examples, the gap is between 0.25 inches and 8 inches.

In some examples, the number of light emitters comprised within the antimicrobial filter layer is based on the distance or gap between the antimicrobial filter layer and the fibrous media filter.

In some examples, the number of light emitters comprised within the antimicrobial filter layer is based on the beam angle of the light emitters and the distance or gap between the antimicrobial filter layer and the fibrous media filter.

In some examples, the antimicrobial filter layer may be designed to fit within air purifiers.

In some examples, the antimicrobial filter layer may be designed to fit within HVAC systems.

In some examples, a control system may turn off the light emitters when a user accesses the antimicrobial filter layer.

In some examples, the light emitters may remain on when a user accesses the antimicrobial filter layer.

In some examples, the fibrous media filter is configured to trap airborne microorganisms.

In some examples, there is no opaque layer or element that may otherwise block light located between the light emitters of the antimicrobial filter layer and the fibrous media filter.

In some examples, the antimicrobial filter layer is static relative to the device it is integrated into when installed and operating.

In some examples, a chamber is built around the antimicrobial filter layer to direct air flow.

In some examples, the device the antimicrobial filter layer is integrated into will comprise a chamber or channels for directing air flow and the antimicrobial filter layer will not direct air flow itself.

In some examples, the antimicrobial filter layer comprises additional structural elements that allow for it to direct air flow.

In some examples, the antimicrobial filter layer is used within a system comprising a fan that forces air flow through the antimicrobial filter layer.

In some examples, the fibrous media filter comprises a photocatalytic agent.

In some examples, the fibrous media filter does not comprise a photocatalytic agent.

In some examples, the antimicrobial filter layer is used within a system that ionizes the air.

In some examples, the antimicrobial filter layer is not used within a system that ionized the air.

In some examples, the fibrous media filter is cylindrical shaped with a hollow center and the antimicrobial filter layer is configured to provide the highest intensity disinfecting light perpendicular to a plane tangent to the interior or exterior cylindrical surface.

In some examples, the number of light emitters and position of light emitters within the antimicrobial filter later are based on the beam angle of the light emitters and the distance between the antimicrobial filter layer and the fibrous media filter. In some examples, the beam angle is between 110 and 150 degrees. In some examples the beam angle is 120 degrees. In some examples, the beam angle is 130 degrees.

We claim:

1. A device comprising:
a pre-filter;
a fibrous media filter;
an antimicrobial filter layer;
wherein the device is configured to allow an airflow to pass through from an entrance point to an exit point; and
wherein the pre-filter is positioned at the entrance point; and
wherein the antimicrobial filter layer is positioned a distance from one or more surfaces of the fibrous media filter and between the entrance point and exit point; and
one or more light emitters positioned within the antimicrobial filter layer and configured to emit a disinfecting light on one or more surfaces of the fibrous media filter.

2. The device of claim 1, further comprising an adsorbent media filter positioned between the entrance point and exit point.

3. The device of claim 1, wherein the disinfecting light comprises an irradiance sufficient to inactivate microorganism on or within the fibrous media filter, and wherein the disinfecting light comprises a wavelength from about 380 nm to about 420 nm.

4. A system comprising:
a fibrous media filter;
an antimicrobial filter layer positioned adjacent to one or more surfaces of the fibrous media filter; and
one or more light emitters positioned within the antimicrobial filter layer and configured to emit a disinfecting light comprising an irradiance sufficient to inactivate microorganisms on or within the fibrous media filter, and wherein the disinfecting light comprises a wavelength from about 380 nm to about 420 nm.

5. The system of claim 4, further comprising a pre-filter.

6. The system of claim 4, further comprising an adsorbent media filter.

7. The system of claim 4, wherein the antimicrobial filter layer comprises gaps configured to allow air to pass through.

8. The system of claim 4, wherein the fibrous media filter is removably attached to the antimicrobial filter layer.

9. The system of claim 4, wherein the fibrous media filter is cuboidal shaped or in a shape of a rectangular prism, and wherein the antimicrobial filter layer is configured to provide the highest intensity disinfecting light perpendicular to one or more sides of the fibrous media filter.

10. The system of claim 4, wherein the fibrous media filter is cylindrical shaped with a hollow center, and wherein the antimicrobial filter layer is configured to provide the highest intensity disinfecting light perpendicular to a plane tangent to the interior or exterior cylindrical surface.

11. The system of claim 4, wherein the antimicrobial filter layer is positioned at a distance from the one or more surfaces of the fibrous media filter, and wherein the distance between the fibrous media filter and the antimicrobial filter layer is at least 0.25 inches.

12. The system of claim 4, wherein the number of light emitters and position of light emitters within the antimicrobial filter later are based on the beam angle of the light emitters and the distance between the antimicrobial filter layer and the fibrous media filter, wherein the beam angle is between 110 and 150 degrees.

13. The system of claim 11, wherein the distance between the fibrous media filter and the antimicrobial filter layer is from 0.25 inches to 8 inches.

14. The system of claim 4, configured for use within an air purification or heating, ventilation, air conditioning (HVAC) device.

15. A method comprising:
providing a fibrous media filter in an air purification or HVAC device;
positioning an antimicrobial filter layer adjacent to one or more surfaces of the fibrous media filter;
embedding one or more light emitters within the antimicrobial filter layer, wherein the one or more light emitters are configured to emit a disinfecting light comprising an irradiance sufficient to inactivate microorganisms, and wherein the disinfecting light comprises a wavelength from about 380 nm to about 420 nm;
illuminating the one or more surfaces of the fibrous media filter with the disinfecting light of the one or more light emitters; and
inactivating microorganisms on or within the fibrous media filter.

16. The method of claim 15, further comprising an airflow from an entrance point to an exit point.

17. The method of claim 16, further comprising providing a pre-filter at the entrance point and adjacent or coupled to the antimicrobial filter layer.

18. The method of claim 17, further comprising providing an adsorbent media filter positioned between the entrance point and exit point.

19. The method of claim 15, wherein the antimicrobial filter layer is positioned at a distance from the one or more surfaces of the fibrous media filter, and wherein the distance between the fibrous media filter and the antimicrobial filter layer is at least 0.25 inches.

20. The method of claim 19, wherein the distance between the fibrous media filter and the antimicrobial filter layer is from 0.25 inches to 8 inches.
